(19) United States
(12) Patent Application Publication
(10) Pub. No.: US 2003/0082201 A1
(43) Pub. Date: May 1, 2003

(54) Multivalent synthetic vaccine for cancer

(75) Inventors: Rama Mukherjee, Ghaziabad (IN); M.R.S. Rao, Bangalore (IN); Arnand C. Burman, Ghaziabad (IN); Becky Thomas, Ghaziabad (IN); Sudhanand Prasad, Ghaziabad (IN); Paromita Sengupta, Ghaziabad (IN)

Correspondence Address: LADAS & PARRY, 26 WEST 61ST STREET, NEW YORK, NY, 10023, US

(73) Assignee: DABUR RESEARCH FOUNDATION

(21) Appl. No.: 10/211,994

(22) Filed: Aug. 2, 2002

Related U.S. Application Data
(60) Provisional application No. 60/309,975, filed on Aug. 3, 2001.

(51) Int. Cl. A61K 39/02; C12Q 1/68; C07H 21/04; C12P 21/02; C12N 1/21; C12N 15/74

(52) U.S. Cl. 424/190.1; 435/6; 435/69.3; 435/252.33; 435/320.1; 530/350; 536/23.2

(57) ABSTRACT

Multivalent vaccine comprising peptides from vasoactive intestinal peptide, bombesin, Substance P and epidermal growth factor are described. A method of constructing a multivalent gene for use in various expressions vectors and the protein recombinantly expressed in the prokaryotic expression systems are also described.

### FIELD OF THE INVENTION

The present invention is in the field of molecular biology, immunology and medicine, and it relates to a synthetically prepared multivalent vaccine for cancer. The vaccine of this invention comprises of a polypeptide which is composed of Vasoactive intestinal peptide (VIP), Bombesin, Substance P and Epidermal growth factor (EGF). The vaccine of this invention comprises the peptides expressing VIP, SP, Bombesin and EGF. The present invention relates to the design and construction of a synthetic polypeptide coupled through linkers. The present invention also relates to the construction of the synthetic gene to the respective designed polypeptide. It also relates to the synthesis of the multivalent gene by Splice-Overlap-Extension PCR. The constructed multivalent gene is then used for cloning in various expression vectors and the protein recombinantly expressed in the prokaryotic expression system. In the present invention the recombinantly expressed protein was also used to study its immunological respone in the murine model.

The invention will be useful as a pharmaceutical composition for the prevention and treatment of various forms of cancers. More particularly, the present work relates to the preparation of human anti-cancer vaccines useful for the prevention and treatment of cancers such as colon, rectum, lung, breast, brain, pancreas, prostrate, liver, gastrointestinal, thyroid, ovary, head and neck, kidney, melanomas, neuro-blastoma, glioblastoma leukemia and lymphomas in mammals, particularly humans.

### DESCRIPTION OF THE PRIOR ART

Cancer vaccines are on the threshold of taking their place alongside the more traditional cancer treatment modalities of surgery, radiation therapy and chemotherapy (Curr Opin Mol Ther, 3(1): 77-84, 2001). The treatment of cancer requires increased tumor protective immunity by active immunization to tumor antigens The epitope-based approach is clearly compelling in the approach to vaccine strategy. A relatively tiny, but immunologically relevant sequence is often capable of inducing protective immunity against large and complex pathogen. (Immunol. And Cell. Biol; 75:402-408, 1997). Scott, A. T., et al. have reported the construction of a recombinant vaccinia virus coding for an artificial polyepitope protein containing 10 contiguous minimal CTL epitopes which were restricted by five MHC alleles and derived from five viruses, a parasite and a tumor model. The construct coding for this protein was capable of inducing MHC-restricted primary CTL responses to all 10 epitopes (J. Immunol, 1196, 157:822-826).

Recent reports reflect that a multi-antigen vaccine for cancer would be advantageous. Firstly, by simultaneously attacking multiple antigen targets, such a vaccine might limit immune evasion by tumors, which have reduced expression of individual target antigen. Secondly, such a vaccine might prime for protective CTL responses directed towards antigens not previously recognized by the immune system.

A common theme in human cancers is the stimulation of tumor cell growth by autocrine and paracrine signaling. In addition to polypeptide growth factors such as EGF, recent evidences supports the role of specific neuropeptides in gastric, colorectal, pancreatic and prostatic cancers. (Oncogene Mar. 26, 2001; 20(13):1563-9). These neuropeptides have defined physiological functions such as neurotransmitters.

Vasoactive intestinal peptide (VIP) is a 28 amino acid neuroendocrine mediator with a broad range of biological activities in diverse cells and tissues. VIP receptors are expressed on adenocarcinomas, breast cancers, melanomas and pancreatic carcinomas. (Cancer Res.; 54, 690-700,1994). Elements within the entire primary sequence are necessary for recognition by the receptors. Today VIP is widely considered to have a physiological regulatory influence on a wide spectrum of body functions, being involved in the pathogenesis of several human disorders and offers hope for the improved prevention or treatment of certain diseases (Immunology Today; 21, No 1; 2000). In children suffering from neuroblastoma, VIP may stimulate or inhibit tumor growth, in an autocrine fashion (Adv. Neuroimmunol, 6: 29-36, 1996). VIP has been shown to possess growth factor effects on mammary cancer, prevalent in adult female population. (Cancer 67: 1561-1564, 1991). VIP1 receptors are present in breast cancer cells. VIP hybrid is a VIP receptor antagonist that inhibits breast cancer proliferation. A VIP analog has been developed for imaging breast tumors. VIP 1 receptors may be utilized for the early detection and treatment of breast cancers (Ann NY Acad. Sci 11, 865: 290-296, 1998). VIP also exhibits differential effects on the invasive potential, rapid progression, and aggressive clinical course of prostate cancer, via the cAMP production (J. Urol. 149:1209-1213; 1993). In many lung cancer cell lines, VIP stimulates mitosis, and the hybrid VIP antagonist inhibits the VIP-stimulated mitosis, both invitro and in vivo, suggesting a possible suggesting role for VIP antagonists in lung cancer inhibition (Biomed Res. 13: 131-135, 1992).

Neuropeptides such as Substance P (SP) and Bombesin regulate many biological processes through binding to and activating their respective cell surface receptors. Many astrocyte/glial-derived brain tumor cell lines express functional SP and bombesin receptors. Activation of these neuropeptide receptors stimulates several signaling pathways that regulate transcription and translation leading to the induction of mitogenesis in several cell types including astrocyte brain tumor-derived cell lines (Int. J. Oncol, 12(2): 273-86 1998).

Bombesin is a tetradecapeptide which typically act as neurotransmitters along the brain-gut axis and as growth factors in various human tissues (J. Am. Soc. Nephrol 2000,1 1(8); 1409-1418). Cancers of the GI tract, breast, lung and prostate frequently express the peptide and its receptors. Most studies have found that bombesin acts to increase tumor cell proliferation, leading to the hypothesis that this peptide hormone is a mitogen important for growth of various cancers (Peptides; 2001: 22(4): 689-99). Malignant transformation of human kidney tissue into renal cell carcinoma is also accompanied by novel expression of the bombesin receptors. Antagonists to bombesin have been proposed as agents for the treatment of cancers. U.S. Pat. No. 6,083,915 refers to a method of treating hepatoma, or liver cancer, in mammalian subjects by administering them with a composition containing a therapeutically effective amount of the bombesin analog. U.S. Pat. No. 5,217,955 refers to a method of treating cancer in a human patient. The method involves administering to the patient analog of a naturally occurring biologically active peptide of mammalian gastrin-releasing peptide, neuromedin B, neuromedin C, bombesin, or litorin. The combination could be used for the treatment of colon, prostate and breast cancer.

Substance P (SP) is a neuropeptide found in both the central and peripheral nervous system. SP is expressed in tumors arising from neural crest derived cells including neuroblastomas, neuro-endocrine carcinomas, medullary carcinomas of the thyroid, etc (J. Cutan Pathol; 25, 2-10, 1998). SP derivatives are potential therapeutic compounds for the treatment of SCLC. [Arg⁶, D-Trp^{7,9,}N^{me}Phe⁸]-substance P (6-11) (antagonist G), a derivative of SP is a novel anti-cancer agent which is entering phase II clinical trials for the treatment of SCLC. (Br. J. cancer 83, 941-948, 2000). SP also have a pro-angiogenic function, stimulates endothelial cell differentiation into capillary-like structures in a dose dependent manner. (Euro. J. Pharmacol 18, 298: 335-338).

EGF is a single polypeptide of 53 amino acid residues, which is involved in the regulation of cell proliferation and mitogenesis. The peptide appears to be non-glycosylated and is very stable (Cell Biology International, Vol. 19, 5, 1996). The biological effect of EGF is mediated by binding of EGF with its specific cell-surface receptors and culminates in biochemical events that lead to DNA synthesis (J. of Cellular Physiol, 148: 220-227, 1991). There is evidence of a relationship between EGF and tumor cell proliferation, such as the overexpression of EGF receptor (EGFR) in different human tumors, which makes this system an interesting target for cancer treatment (Annal. Oncol 9,431-5, 1998). In a study carried out in Philadelphia, USA, a naturally occurring mutant human EGF receptor (EGFRvIII) was tried as a target for peptide vaccine immunotherapy of tumors (Cancer Res. 15; 57(8); 1419-24, 1997). This spontaneously occurring alteration is found in a high percentage of primary human brain, breast, lung and ovarian tumors. Pre immunization of mice with this peptide substantially increased tumor formation of cells expressing EGFRvIII. Thus, the alteration present in EGFRvIII could serve as the basis for an anti-tumor vaccine with potentially wide applications in humans. Growth inhibitory action of EGF has been reported in epidermal carcinoma A431 cells, which have an unusually high density of cell surface EGF receptors (Cell Growth Differ 7(2): 173-8, 1996). U.S. Pat. application No. 5,894,018, refers to a vaccine composition comprising autologous EGF coupled to a carrier protein such as tetanus toxoid or cholera toxin B chain. An important objective of this invention was to obtain a vaccine composition for the active immunotherapy of EGF dependent malignant tumors, for, e.g. Epidermal carcinoma of lung, glioblastoma multiforme and head and neck epidermoid carcinoma.

U.S. Pat. No. 6,156,725, refers to the use of a combination of peptides, viz, Somatostatin, bombesin, VIP and substance P to block the uncontrolled multiplication of cancer cells of the colon, rectum, lung, breast, and kidney. The invention also relates to a pharmaceutical composition containing a combination of such analogs. The combination may also be useful in preventing, inhibiting, or modulating the hypersecretion of VIP, bombesin, substance P or a combination of all.

### REFERENCES

1. Emens L A, et al: Chemotherapy: friend or foe to cancer vaccines? Curr Opin Mol Ther, 3(1): 77- 84, (2001).

2. Suhrbier, A.: Multi-epitope DNA vaccines; Immunology and Cell Biology, 75: 402-408 (1997).

3. Thomson, A. S., et al.: Recombinant Polypeptide Vaccines for the delivery of Multiple CD8 Cytotoxic T Cell Epitopes: J. Immunol, 157: 822-826 (1996).

4. Heasley, L. E.: Autocrine and Paracrine signalling through neuropeptide receptors in human cancer: Oncogene, 20 (13):1563-9 (2001).

5. Virgolini, I., et al.:Cross-Competition between Vasoactive Intestinal Peptide and Somatostatin for binding to Tumor Cell membrane receptors.: Cancer Research, 54: 690-700 (1994).

6. Pozo, D., et al.: Immunobiology of vasoactive intestinal peptide.: Immunology Today, 21, 1(2000).

7. Leceta, J. et al.: Expression of vasoactive intestinal peptide in lymphocytes—a possible endogenous role in the regulation of the immune system: Adv. Neuroimmunol, 6(1):29-36, (1996).

8. Scholar, E. M. & Paul, S.:Stimulation of tumor cell growth by vasoactive intestinal peptide.: Cancer, 67: 1561-1564, (1991).

9. Gozes, I., et al.: Neuropeptide Regulation of Mitosis: Ann NY Acad. Sci, 11 865: 290-296, (1998).

10. Hoosein, N. M.: Differential effects ofpeptide hormones bombesin, vasoactive intestinal peptide and somatostatin analog RC-160 on invasive capacity of human prostatic carcinoma cells:J. Urol, 149:1209-1213, (1993).

11. Moody, T. W., et al.:VIP analogues inhibit small cell lung cancer growth: Biomed Res.13:131-135 (1992).

12. Sharif, M.: Mitogenic signalling by substance P and bombesin-like neuropeptide receptors in astrocyte/glial brain tumor-derived cell lines: Int. J. Oncol. 12(2): 273-86, (1998).

13. Pansky, A., et al: Gastrin releasing peptide-preferring bombesin receptors mediate growth of human renal cell carcinoma:.J. Am. Soc. Nephrol,1 1(8); 1409-1418(2000).

14. Jensen, J A., et al: The case for gastrin-releasing peptide acting as a morphogen when it and its receptor are aberrantly expressed in cancerPeptides: 22(4): 689-99(2001).

15. Khare, K. V.: The neuropeptide/mast cell secretagogue substance P is expressed in cutaneous melanocytic lesions:J. Cutan Pathol; 25, 2-10, (1998).

16. Mac Kinnon, A C. et al [Arg⁶, D-Trp^{7,9,}N^{me}Phe⁸]-substance P (6-11) (antagonist G) induces AP-1 transcription and sensitizes cells to chemotherapy: Br. J. cancer 83, 941-948, (2000).

17. Wiedermann, C J. et al.: Induction of endothelial cell differentiation into capillary-like structures by substance P: Euro. J. Pharmacol 18, 298: 335-338 (1996).

18. Boonstra, J. et al.: The Epidermal growth Factor: Cell Biology International, Vol. 19, 5, (1996).

19. Huang, S. et al:Growth modulation by epidermal growth factor (EGF) in human colonic carcinoma cells: constitutive expression of the human EGF gene: J. of Cellular Physiol; 148: 220-227, 1(991).

20. Gonzalez G. et al: A novel cancer vaccine composed of human-recombinant epidermal growth factor linked to a carrier protein: report of a pilot clinical trial: Annal. Oncol 9,431-5, (1998).

21. Moscatello D K, et al: A naturally occuring mutant epidermal growth factor receptor as a target for peptide vaccine immunotherapy of tumors: Cancer Res. 15; 57(8); 1419-24, (1997).

22. Gulli L F, et al: Epidermal growth factor-induced apoptosis in A431 cells can be reversed reducing the tyrosine-kinase activity: Cell Growth Differ 7(2): 173-8, (1996).

### SUMMARY OF THE INVENTION

The present invention describes the design of the polypeptide consisting of the four peptides viz. Vasoactive Intestinal Peptide (VIP) (SEQ ID NO: 1), Bombesin (Bom) (SEQ ID NO: 2), Substance P (SP) (SEQ ID NO: 3) and Epidermal Growth Factor (EGF) (SEQ ID NO: 4); which are covalently linked with or without linkers and may be useful as vaccine for the treatment of cancer. The preferred designed polypeptide has a total of 112 aminoacids as described in (SEQ ID NO: 5) in which the four mentioned peptides are linked through Gly-Gly linker. The present invention also relates to the design of corresponding synthetic gene using codon optimization for the prokaryotic system and the sequence of the designed synthetic gene is described in SEQ ID NO: 6. It also describes the method for the construction of complete multivalent synthetic gene, its cloning in various prokaryotic vectors and expression in the E. coli system. The method of purification and characterization of the expressed recombinant protein has also been described in this patent application. It also describes production of polyclonal antibodies and T-cell immune response in the murine model, using the recombinantly expressed protein.

The recombinant protein has been synthesized with the presumption that a multi-epitope vaccine could induce immunity against multi antigenic targets and hence could be used for the treatment of a broad spectrum of cancers. This present invention could prove helpful in preventing, inhibiting, or modulating the hypersecretion of VIP, bombesin, substance P and EGF or a combination of all.

### BRIEF DESCRIPTION OF FIGURES

FIG. 1: shows the schematic depiction of the PCR method to be followed in the construction of the synthetic multivalent vaccine using the oligonucleotides depicted in SEQ ID NO: 7-14.

FIG. 2: schematically depicts the strategy to be followed for the cloning and expression of the constructed gene in appropriate vectors.

FIG. 3: SOE in PCR of Multivalent gene. After each PCR, the product was gel eluted (using Qiagen Kit) and used as the template for the next-step of PCR. Lane 1; 100 bp ladder; Lane 2: 126 bp DNA (after first step-PCR); Lane 3: 179 bp DNA (after second-step PCR); Lane 4: 232 bp DNA (after third-step PCR); Lane 5: 285 bp DNA (after fourth-step PCR); Lane 6: 336 bp DNA (after fifth step PCR); Lane 7: 336 bp DNA (after final step PCR); Lane 8: 100 bp ladder.

FIG. 4: shows the vector map of pET-22b(+).

FIG. 5: shows the vector map of pGEX 5×2.

FIG. 6: shows the vector map of pPRSET A.

FIG. 7: Cloning in pET-22b(+)-Screening of positive clones by restriction enzyme digestion. Plasmid DNA isolation of the clones was done by Alkaline SDS Method. For identifying the positive clones, plasmid DNA was digested with the restriction enzymes EcoR1 and Not 1. The digested sample was run on a 1.8% analytical agarose gel. Lane 1-6: mini prep samples; Lane 7: 100 bp ladder.

FIG. 8: depicts the nucleotide sequence of clone #2 pET-MV (SEQ ID NO: 18). FIG. 8 (SEQ ID NO: 18) depicts the nucleotide sequence of the clone pET MV#2. The highlighted nucleotides e.g. “g” and “a” (in bold, underlined and italicized) show the substitutions that have taken place in this clone, as compared to the original designed multivalent nucleotide sequence as depicted in SEQ ID NO: 6

FIG. 9: depicts the amino sequence of clone #2 pET-MV (SEQ ID NO: 19). FIG. 9 (SEQ ID NO: 19) depicts the aminoacid sequence of the clone pET MV#2, which has been extrapolated from the corresponding nucleotide sequence (FIG. 8; SEQ ID NO: 18), using the translational tool software. The highlighted aminoacid e.g. “Gly” and “Ser” (in bold, underlined and italicized) show the substitutions that have taken place in this clone, as compared to the original designed multivalent aminoacid sequence as depicted in SEQ ID NO: 5.

FIG. 10: Cloning in pGEX5×2-Screening of positive clones by restriction enzyme digestion. Plasmid DNA isolation of the clones was done by Alkaline SDS Method. For identifying the positive clones, plasmid DNA was digested with the restriction enzymes EcoR1 and Not 1. The digested sample was run on a 1.8% analytical agarose gel. Lane 1-7: mini prep samples; Lane 8: 100 bp ladder.

FIG. 11: depicts the nucleotide sequence of clone #16 pGEX-MV (SEQ ID NO: 20). FIG. 11 (SEQ ID NO: 20) depicts the nucleotide sequence of the clone pGEX MV#16. The clone shows 98% homology with the original designed multivalent nucleotide sequence (SEQ ID NO: 6). The reading frame of the clone has been maintained till the 95^{th} nucleotide codon. At the 95^{th} codon, there is an addition of a nucleotide “t”, followed by three substitutions viz., “a”, “g” and “t”(in bold, underlined and italicized). Consequently, there has been a reading frame-shift, leading to an inclusion of a stop codon viz. “tga” at the 99^{th} codon position.

FIG. 12: depicts the amino sequence of clone #16 pGEX-MV (SEQ ID NO: 21). FIG. 12 (SEQ ID NO: 21) depicts the aminoacid sequence of the clone pGEX MV #16, which has been extrapolated from the corresponding nucleotide sequence (FIG. 11; SEQ ID NO: 20), using the translational tool software. The changes in the aminoacid sequence have been highlighted. Due to the incorporation of a stop codon, the last 14 amino acids are not expressed. Also, the 3 amino acids before the stop codon have been altered due to the substitutions and additions at the nucleotide level.

FIG. 13: Cloning in pET-22b(+)[incorporating a stop codon]-Screening of positive clones by restriction enzyme digestion. Plasmid DNA isolation of the clones was done by Alkaline SDS Method. For identifying the positive clones, plasmid DNA was digested with the restriction enzymes EcoR1 and Not 1. The digested sample was run on a 1.8% analytical agarose gel. Lane 1-7: mini prep samples; Lane 8: 100 bp ladder.

FIG. 14: depicts the nucleotide sequence of clone #1 pET-MVSC (SEQ ID NO: 22). FIG. 14 (SEQ ID NO: 22) depicts the nucleotide sequence of the clone pET MVSC#1. The highlighted nucleotide e.g. “g”(in bold, underlined and italicized) show the substitution that has taken place in this clone, as compared to the original designed multivalent nucleotide sequence as depicted in SEQ ID NO: 6. Since the substitution was at the 3^{rd} position in the codon, the amino acid coded by the codon remained.

FIG. 15: depicts the amino acid sequence of clone#1 pET-MVSC (SEQ ID NO: 23). FIG. 15 (SEQ ID NO: 23) depicts the aminoacid sequence of the clone pET MV SC #1, which has been extrapolated from the corresponding nucleotide sequence (FIG. 14; SEQ ID NO: 22), using the translational tool software. The aminoacid sequence shows 100% homology to the original designed multivalent aminoacid sequence as depicted in SEQ ID NO: 5.

FIG. 16: depicts the nucleotide sequence of clone #9 pET-MVSC (SEQ ID NO: 24). FIG. 16 (SEQ ID NO: 24) depicts the nucleotide sequence of the clone pET MVSC #9. The clone showed 100% homology with the original designed multivalent nucleotide sequence as depicted in SEQ ID NO: 6

FIG. 17: depicts the amino acid sequence of clone #9 pET-MVSC (SEQ ID NO: 25). FIG. 17 (SEQ ID NO: 25) depicts the aminoacid sequence of the clone pET MV SC #9, which has been extrapolated from the corresponding nucleotide sequence (FIG. 16, SEQ ID NO: 24), using the translational tool software. The aminoacid sequence shows 100% homology to the original designed multivalent aminoacid sequence as depicted in SEQ ID NO: 5.

FIG. 18: depicts the nucleotide sequence of clone #14 pET-MVSC (SEQ ID NO: 26). FIG. 18 (SEQ ID NO: 26) depicts the nucleotide sequence of the clone pET MVSC #14. The highlighted nucleotides e.g. “t” (in bold, underlined and italicized) shows the substitution that has taken place in this clone, as compared to the original designed multivalent nucleotide sequence as depicted in SEQ ID NO: 6

FIG. 19: depicts the amino acid sequence of clone #14 pET-MVSC (SEQ ID NO: 27). FIG. 19 (SEQ ID NO: 27) depicts the aminoacid sequence of the clone pET MV SC #14, which has been extrapolated from the corresponding nucleotide sequence (FIG. 18, SEQ ID NO: 26), using the translatioin tool software. The highlighted aminoacid e.g. “Glu” (in bold, underlined and italicized) shows the substitution that has taken place in this clone, as compared to the original designed multivalent aminoacid sequence as depicted in SEQ ID NO: 5

FIG. 20: SDS-PAGE profile of expression of pET-MV2 at 30° C.: pET-MV2 was expressed in BL21DE3λ. The culture was induced with 1 mM IPTG at 30° C. and expression of the protein was studied at various time points. At each time point, the induced and uninduced aliquots (1-ml) of the culture were pelleted down. The pellet was boiled in 5×SDS-PAGE loading dye, boiled for 10 minutes and 15 ul of the sample was loaded on a 15% SDS-PAGE. Lane 1: Induced sample post 2 hour of IPTG addition; Lane 2: Uninduced sample post 2 hour; Lane 3: Induced sample post 2_{½} hour of IPTG addition; Lane 4: Uninduced sample post 2_{½} hour.

FIG. 21: SDS-PAGE profile of expression of pET-MV2 at 37° C.: pET-MV2 was expressed in BL21DE3λ. The culture was induced with 1 mM IPTG at 37° C. and expression of the protein was studied at various time points. At each time point, the induced and uninduced aliquots (1-ml) of the culture were pelleted down. The pellet was boiled in 5×SDS-PAGE loading dye, boiled for 10 minutes and 15 ul of the sample was loaded on a 15% SDS-PAGE. Lane 1: Uninduced sample post 2 hour of IPTG addition; Lane 2: Induced sample post 2 hour; Lane 3: UnInduced sample post 2_{½} hour of IPTG addition; Lane 4: Induced sample post 2_{½} hour. Lane 5: molecular weight marker.

FIG. 22: Immunoblot of pET-MV2: pET-MV2 was expressed in BL21DE3λ. The culture was induced with 1 mM IPTG at 30° C. After 2_{½} hour of IPTG induction, cells were pelleted down. The pellet was boiled in 5×SDS-PAGE loading dye, boiled for 10 minutes and 15 μl of the sample was loaded on a 15% SDS-PAGE. The proteins were electrophoretically transferred to a nitrocellulose membrane. The membrane was blocked with 3% BSA and probed with anti-peptide antibodies. Lane 1: Immunoblot of induced crude extract with anti-VIP antibody; Lane 2: Immunoblot of crude extract with anti-His antibody; Lane 3: Immunoblot of induced crude extract with anti-SP antibody; Lane 4: Immunoblot of induced crude extract with anti-EGF; Lane 5: Immunoblot of uninduced crude extract with anti-SP antibody.

FIG. 23: Immunoblot of purified multivalent protein: The crude extract of the induced pET-MV2 clone was loaded on a Ni-NTA column. Th protein was eluted with increased concentration of imidazole. The protein samples were loaded on 15% SDS-PAGE and electrophoretically transferred to a nitrocellulose membrane. The membrane was blocked with 3% BSA and probed with anti-SP antibody. Lane 1: Crude extract; Lane 2: Purified fraction; Lane 3: Purified fraction.

FIG. 24: SDS-PAGE of gel purified multivalent protein: The crude extract of the induced pET-MV2 clone was loaded on 15% SDS-PAGE. Electrophoresis was done at constant voltage of 100V for about 3 hrs. As described in the example, the portion of the gel corresponding to the desired protein was cut, washed with 2 mM Phosphate buffer (pH 6.8) minced and put in a dialysis tubing (Mol wt. Cut off-3,500 Da). Electroelution was carried out at 40 V (for 40 min at 4° C.) in a buffer tank containing 2 mM Phosphate buffer (pH 6.8). The protein sample was taken out from the dialysis bag and was concentrated in a speed vac, under cold conditions. It was reconstituted in minimal volume of 2 mM PBS and loaded on a 15% SDS-PAGE gel. Lane1: Mol wt marker, Lane3: Eluted protein; Lane 4: Crude extract.

FIG. 25: Immunoblot of gel purified multivalent protein: The electroeluted protein was loaded on a 15% SDS-PAGE gel. The proteins were then electrophoretically transferred to a nitrocellulose membrane, blocked with 3% BSA and probed with anti-peptide antibodies. FIG. 25 A. Immunoblot with anti-SP antibody Lane 1: Crude extract; Lane 2: Electroeluted protein FIG. 25. B. Immunoblot with anti-VIP antibody Lane 1: Crude extract; Lane2:Electroeluted protein.

FIG. 26: SDS-PAGE profile of expression of pGEX-MV16 at 30° C.: pGEX MV16 was expressed in BL21DE3λ. The culture was induced with 1 mM IPTG at 30° C. and expression of the protein was studied at various time points. At each time point, the induced and uninduced aliquots (1-ml) of the culture were pelleted down. The pellet was boiled in 5×SDS-PAGE loading dye, boiled for 10 minutes and 15 ul of the sample was loaded on a 15% SDS-PAGE. Simultaneously as control, the vector alone (pGEX 5×2) was induced and expressed. Lane 1: Uninduced sample of pGEX 5×2 post 2 hour of IPTG addition; Lane 2: Induced sample post 2 hour of pGEX 5×2; Lane 3: Induced sample of pGEX. MV16 post 2 hour IPTG addition; Lane 4: UnInduced samples of pGEX MV16 post 2 hour IPTG addition; Lane 5: Induced Sample of PGEX-5×2.

FIG. 27: SDS-PAGE profile of expression of pGEX-MV16 at 37° C.: pGEX MV16 was expressed in BL21DE3λ. The culture was induced with 1 mM IPTG at 37° C. and expression of the protein was studied at various time points. At each time point, the induced and uninduced aliquots (1-ml) of the culture were pelleted down. The pellet was boiled in 5×SDS-PAGE loading dye, boiled for 10 minutes and 15 ul of the sample was loaded on a 15% SDS-PAGE. Simultaneously as control, the vector alone (pGEX 5×2) was induced and expressed. Lane 1: Induced sample of pGEX_MV16 post 2 hour of IPTG addition; Lane 2: Uninnduced sample of pGEX_MV16 post 2 hour of IPTG addition; Lane 3: Induced sample of pGEX 5×2 post 2 hour IPTG addition; Lane 4: Uninduced sample of pGEX 5×2 post 2 hour IPTG addition.

FIG. 28: Immunoblot of pGEX-MV16: pGEX-MV16 was expressed in BL21DE3λ. The culture was induced with 1 mM IPTG at 30° C. After 2 hour of IPTG induction, cells were pelleted down. The pellet was boiled in 5×SDS-PAGE loading dye, boiled for 10 minutes and 15 μl of the sample was loaded on a 15% SDS-PAGE. The proteins were electrophoretically transferred to a nitrocellulose membrane. The membrane was blocked with 3% BSA and probed with anti-VIP antibody. Lane 1: Uninduced pellet of pGEX MV16; Lane 2: Induced pellet of pGEX MV16; Lane 3: Uninduced pellet of pGEX Lane 4: Induced pellet of pGEX.

FIG. 29: SDS-PAGE profile of purified fractions of pGEXMV16: pGEX MV16 was expressed in BL21DE3λ. The fusion protein was purified on Glutathione-sepharose column. Lane1: Molecular weight marker; Lane 2-8: Purified fractions

FIG. 30: Immunoblot of purified fractions of pGEXMV16: pGEX MV16 was expressed in BL21DE3λ. The fusion protein was purified on Glutathione-sepharose column. The purified fractions were loaded on a 15% SDS-PAGE. The proteins were electro-phoretically transferred to a nitrocellulose membrane. The membrane was blocked with 3% BSA and probed with anti-SP antibody. Lane 1: Uninduced pellet of pGEX MV16; Lane 2: Induced pellet of pGEX MV16; Lane 4-8: Purified fractions.

FIG. 31: SDS-PAGE profile of expression of pET-MVSC1 at 30° C.: pET-MVSC1 was expressed in BL21DE3λ. The culture was induced with 1 mM IPTG at 30° C. and expression of the protein was studied at various time points. At each time point, the induced and uninduced aliquots (1-ml) of the culture were pelleted down. The pellet was boiled in 5×SDS-PAGE loading dye, boiled for 10 minutes and 15 ul of the sample was loaded on a 15% SDS-PAGE. Lane 1: Induced sample post 2 hour of IPTG addition; Lane 2: Uninduced sample post 2 hour; Lane 3: Induced sample post 2_{½} hour of IPTG addition; Lane 4: Uninduced sample post 2_{½} hour.

FIG. 32: SDS-PAGE profile of expression of pET-MVSC14 at 30° C.: pET-MVSC1 was expressed in BL21DE3λ. The culture was induced with 1 mM IPTG at 30° C. and expression of the protein was studied at various time points. At each time point, the induced and uninduced aliquots (1-ml) of the culture were pelleted down. The pellet was boiled in 5×SDS-PAGE loading dye, boiled for 10 minutes and 15 ul of the sample was loaded on a 15% SDS-PAGE. Lane 1: Induced sample post 2 hour of IPTG addition; Lane 2: Uninduced sample post 2 hour; Lane 3: Induced sample post 2_{½} hour of IPTG addition; Lane 4: Uninduced sample post 2_{½} hour.

FIG. 33: Polyclonal antibody titers to recombinantly expressed multivalent protein and individual peptides: 96-well ELISA plates were coated with 500 ng/well of the multivalent protein and 500 ng/well of Bombesin, EGF, Substance P and VIP respectively. The non-specific sites were blocked with 3% BSA and subsequently probed with 1:100 dilution of pre-immune serum, boost1 and boost 2 sera respectively. Color was developed with o-phenylenediamine (OPD), and optical density read at 492 nm.

FIG. 34: Immunoblot of tumor lysates with polyclonal antibodies: Tumor cell lysates of breast cancer (MCF-7) and prostate cancer (DU145) were probed with either rat polyclonal antibodies (preimmune or boost2 sera) to the recombinantly expressed multivalent protein (Lane 1, 2 and 3), or with anti-SP antibodies (Lane 4 and 5) respectively

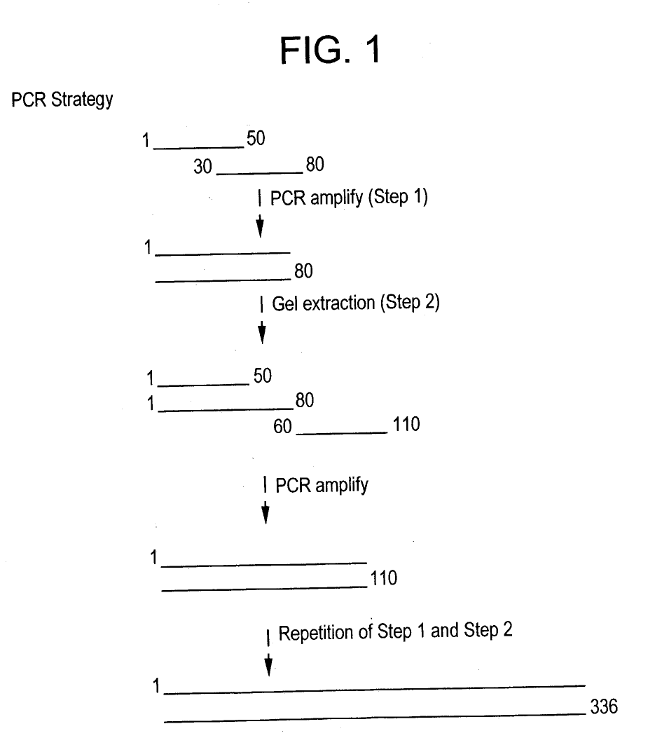

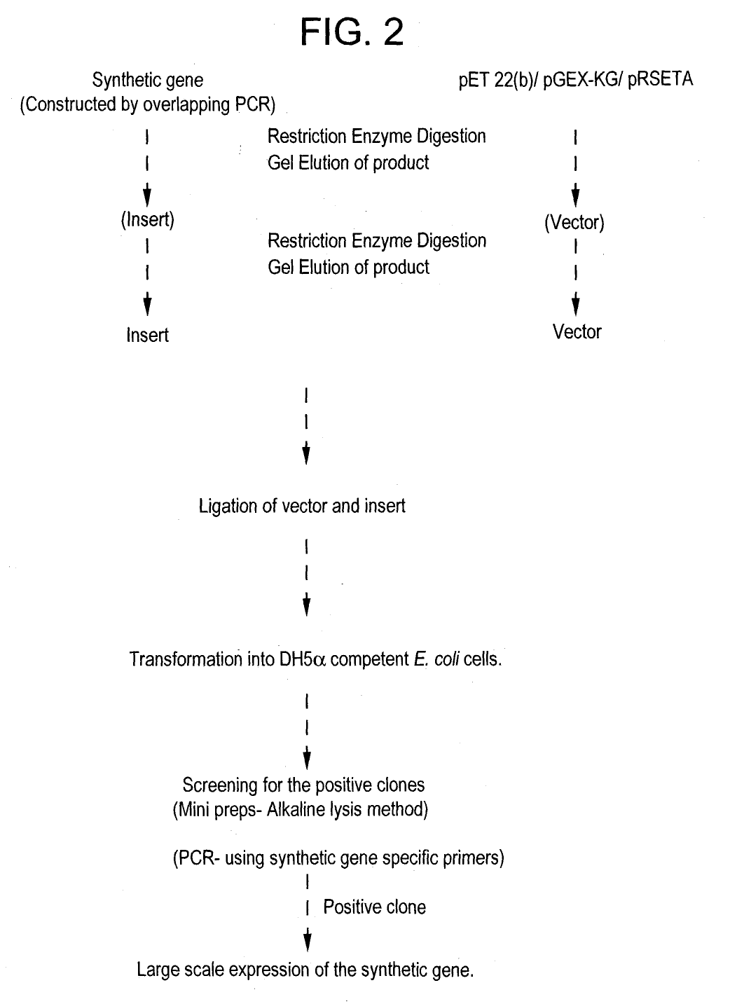

### DETAILED DESCRIPTION OF INVENTION

The present invention relates to a synthetic multivalent epitope vaccine. For the synthesis of this multivalent vaccine, we identified four peptides viz. VIP, Bombesin, Substance P and EGF, which are overexpressed in cancer. The immuno-genic epitopes of each of the four peptides were identified and their aminoacid sequence determined. A multivalent polypeptide sequence was designed with a two aminoacid viz. Glycine-Glycine as linker between each of the four peptides. Following the design of the polypeptide sequence, they were backtranslated into the nucleotide sequence. For this, the prokaryotic codon optimization was used to enable optimal cloning and expression of the multivalent peptide in E. coli. Based on the designed multivalent gene sequence, oligonucleotides were synthesized for Splice-Overlap-Extension (SOE) PCR. 70-mer oligonucleotides were synthesized with an overlap region of 20-mer. As described below in the Examples of this invention, the gene coding for synthetic multivalent vaccine was constructed by overlapping PCR technology using oligonucleotides corresponding to the sequence of the designed gene. After each step of PCR, the gene product was purifed by gel-elution kit and gene sequencing done to confirm the correct sequence. The complete amplified constructed gene was then cloned into various prokaryotic expression vectors and expressed in the E. coli expression system. The protein expression was visualized by SDS-PAGE and the multivalent protein migrated at a molecular weight of 11 kDa. The multivalent protein was probed with commercially available antibodies to each of the individual peptides by Western blot, to confirm the presence of the respective peptides in the multivalent sequence. The recombinantly expressed protein was then purified using affinity column chromatography/electroelution/standard chromatographic procedures. The expressed multivalent protein was further used to immunize rats for the production of polyclonal antibodies. The multivalent protein was also studied for its possible role in elucidating a CMI/T-cell response in the murine model.

The bacterial plasmid vectors which was used for the cloning of the multivalent synthetic vaccine includes the E. coli plasmids viz. pGEX, pET, etc. and their derivatives. The pET vectors are derived from pBR322 and vary in leader sequences, expression signals, relevant restriction sites, etc. These vectors are classified as the transcriptional and translational vectors. The translational vectors, such as pET-20, pET-22, pET-25, etc. contain efficient translational signals and are designed for protein expression. Target genes are cloned under control of strong bacteriophage T7 transcription and translational signals, and providing a source of T7 RNA polymerase in the host cell induces expression. The vector has an ampicillin resistance marker. The gene is expressed in frame with His residues, which enables one-step purification of the protein by affinity column using Ni-NTA matrix.

The pGEX vectors are widely used for the cloning and expression of genes. The genes are cloned under the control of tac promoter, which is chemically inducible and allows high level expression of the cloned genes. The gene has been expressed in fusion with the GST protein and thus, can be purified on a glutathione sepharose.

This is one example of a vector that can be used for expression of the multivalent gene and derivatives thereof. Different vectors with various promoters and polyA signals, recombinant viruses, expression vectors for other species such as for example yeast, fungi, insect cells and plants could be used. A selection of large number of prokaryotic hosts, such as E. coli (HM101, BL21, etc.) was used for the expression of the synthetic multivalent gene.

The multivalent protein may be administered serially or in combination with other therapeutics used in the treatment of cancer and other related diseases. These therapeutics include IFN-alpha, IFN-beta, interleukin-1, interleukin-2, tumor necrosis factor, macrophage colony stimulating factor, macrophage activation factor, lympho-toxin, fibroblast growth factor, etc (derived from natural sources or expressed recombinantly). Alternatively, the synthetic multivalent vaccine may be administered serially or in combination with conventional chemotherapeutic agents such as 5-fluoro uracil; paclitaxel; etoposide; carboplatin; cisplatin; topotecan, methatroxate, etc. and/or radiotherapy. Such combination therapies may advantageously utilize less than conventional dosages of those agents, or involve less radical regimens, thus avoiding any potential toxicity or risks associated with those therapies.

As per this invention, the recombinantly expressed multivalent vaccine can be administered in any pharmaceutically acceptable form, intratumorally, peritumorallly, interlesionally, intravenously, intramuscularly, subcutaneously or by topical routes to exert local therapeutic effects. As an alternative to administering the synthetic multivalent vaccine, the gene encoding the vaccine may be introduced into the cancer cells by treating the infected cells, for example, by scraping them to allow uptake of DNA, by electroporation, by direct injection, etc.

For a better understanding of the invention, the following examples are set forth. Throughout these examples, all molecular cloning reactions was carried out according to methods in Molecular Cloning—A laboratory Manual, 3^{rd} Ed., Cold Spring Harbor Press 2001.

### Reagents & Materials

The following chemicals were used for the experiments—Agarose; Ampicillin; Bovine Serum Albumin; Brilliant blue R250; Bromo phenol blue; Diaminobenzidiamine; Sodiumdodecylsulphate; Glucose; Glycine (Sigma); Ethylenediaminetetraaceticacid; Imidazole; Isopropylthiogalactoside (Calbiochem); Phenylmethylsulfonylfluoride; Sodium dihydrogen phosphate; Disodium hydrogen phosphate; Tris (hydroxymethyl aminomethane); Triton-X-100; Tween20; Urea (Merck); Yeast Extract. Tryptone (Difco); Protease inhibitor tablets (Roche). Goat anti-rat-IgG (HRP-labeled) (SIGMA); Deoxycholate; Q-Sepharose (Amersham Pharmacia).

The reagents used for the experiments were procurred from-Anti-EGF antibodies (Sigma, USA; Roche, Germany; Bachem, Switzerland; R&D, USA); Anti-VIP antibodies(Sigma, USA; Bachem, Switzerland); Anti-SP antibodies (Sigma, USA; Bachem, Switzerland; Roche, Germany); Anti-Bombesin (Bachem, Switzerland); Anti-His antibodies (Sigma, USA); Anti-GST antibodies (Sigma, USA); Anti-mouse antibodies (Sigma, USA); Anti-rabbit antibodies (Sigma, USA); Dialysis tubing (Pierce, USA); Glutathione Sepharose (Amersham Pharmacia, UK); Ni-NTA Agarose (Qiagen, USA); Nitrocellulose membrane (Promega, USA); DH5\acute{α} cells (Bangalore Genei); BL21DE3λ cells (Novagen, USA); Restriction digestion enzymes and Modifying enzymes(GIBCO BRL, USA; NEB,UK Promega, USA).

The equipments used for the experiments were—PCR Machine (Perklin Elmer); Incubator shaker (Innova); Tabletop centrifuge (Herraeus); Protein PAGE Apparatus (Bio-Rad); Horizontal gel apparatus (Bangalore Genei); ELISA Reader (Anthos); Spectrophotometer (Shimadzu,).

### EXAMPLE 1

### Construction of the Synthetic Gene

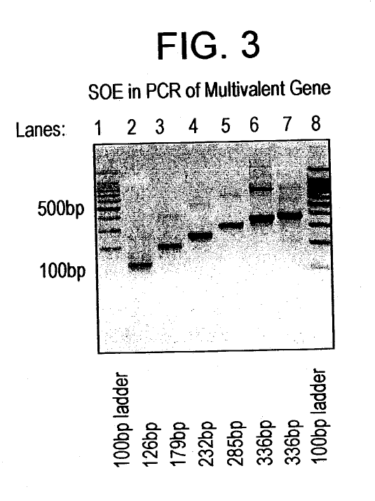

To construct the synthetic gene, 70-mer oligonucleotide have been designed with an overlap of 20-25 mer. In the first step of the PCR, MVSV1 (SEQ ID NO: 7) and MVSV2 (SEQ ID NO: 8) were used. The two primers have an overlap of 20 oligonucleotides. The reaction condition of the PCR was −97° C. for 3 min, followed by thirty cycles of 95° C. at 1 min, 45° C. for 2 min, 72° C. for 30 sec. After this a final step of elongation at 72° C. for 7 min was followed by cooling at 4° C. A 126 bp product (as shown in FIG. 3, lane 2) was obtained after the first step. This amplified product was gel eluted (using Qiagen Kit) and used as the template for the second-step PCR.

In the second step, MVSV7 (SEQ ID NO: 13) and MVSV3 (SEQ ID NO: 9) were used as the forward and reverse primers respectively. Product of the first step PCR was used as the template. The PCR conditions were similar to the first step, except for the annealing temperature (55° C. for 2 min). A 179-bp product was obtained after second-step PCR, (as shown in FIG., 3, lane 3), which was then similarly gel-purified and used as the template for the next step.

In the third step, MVSV7 (SEQ ID NO: 13) and MVSV4 (SEQ ID NO: 10) were used as the forward and reverse primers respectively. Product of the second step PCR was used as the template. The PCR conditions were similar to the first step, except for the annealing temperature (59° C. for 2 min). A 232-bp product was obtained following third-step PCR (as shown in FIG. 3, lane 4), which was then gel purified and used as the template for the following step.

In the fourth step, MVSV7 (SEQ ID NO: 13) and MVSV5 (SEQ ID NO: 11) were used as the forward and reverse primers respectively. Product of the third step PCR was used as the template. The PCR conditions were similar to the third step. 285 bp product was obtained (as shown in FIG. 3, lane 5), which was gel purified and used as the template for the next step.

In the fifth step, MVSV7 (SEQ ID NO: 13) and MVSV6 (SEQ ID NO: 12) were used as the forward and reverse primers respectively. Product of the fourth step PCR was used as the template. The PCR conditions were similar to the first step, except for the annealing temperature (65° C. for 2 min). 336 bp product was obtained (as shown in FIG. 3, lane 6), which was purified and used as the template for the next step.

The sixth PCR step was performed with the primers MVSV7 (SEQ ID NO: 13) and MVSV8 (SEQ ID NO: 14). The primers were of shorter length. This step was performed to obtain a final PCR product, which can then be subsequently used for cloning procedures. The 336 bp PCR product was used as the template. The PCR conditions were similar to the fifth step, except for the annealing temperature (62° C. for 2 min). 336 bp product was obtained (as shown in FIG. 3, lane 7), which was purified and used for next step.

For further cloning of the constructed gene, restriction enzyme sites were incorporated at the 5′ and 3′ ends in the next step of PCR. MVSV9 (SEQ ID NO: 15, having EcoR1 site) and MVSV10 (SEQ ID NO: 16, having Not 1 site) were used. PCR conditions were similar as reported in earlier paragraph. 336-bp product was obtained, which was purified and used for next step. Downstream primer with a stop codon (MVSV 11; SEQ ID NO: 17) was designed, which enabled the cloning of the multivalent gene in pET vector without the fusion tag

### EXAMPLE 2

### Description of the Vectors

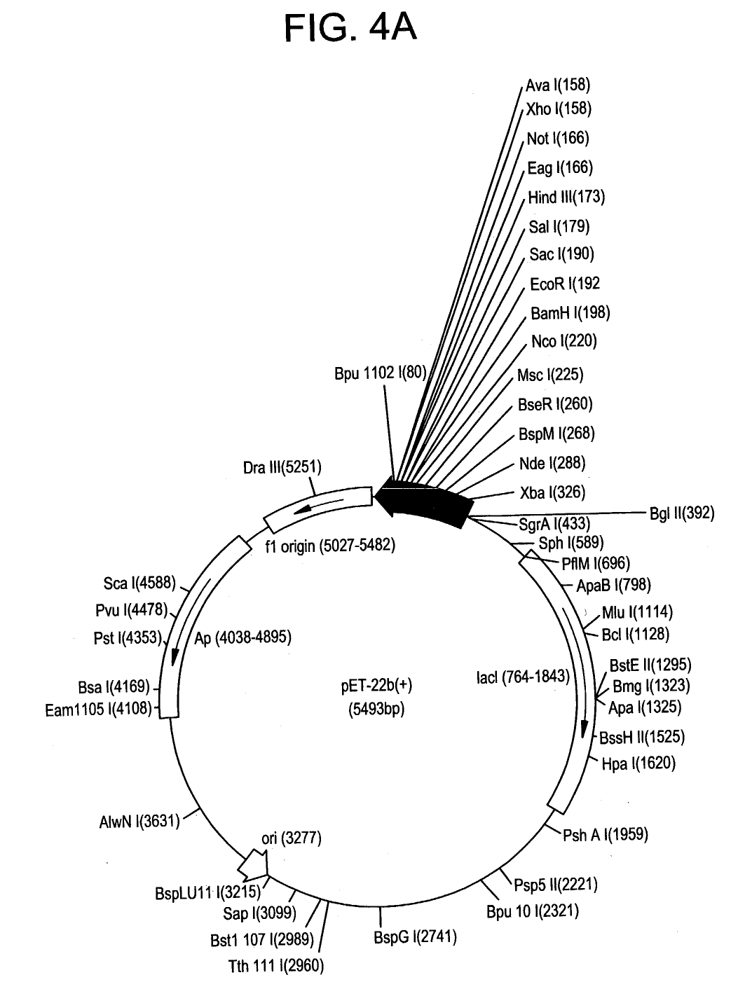

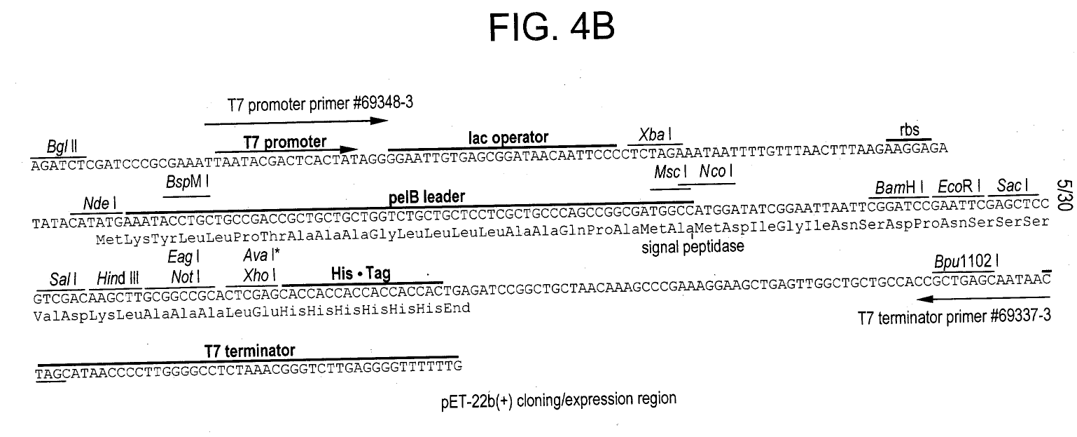

The prokaryotic expression vectors selected for the cloning of the multivalent gene were pET-22b(+), pGEX5×2 and pRSET-A. pET 22b(+) is a 5.5 kb prokaryotic vector. The vector has an Ampicillin resistance marker and a multiple cloning site, which allows the selection of appropriate restriction enzyme sites for cloning (FIG. 4). The target genes are cloned under control of strong bacteriophage T7 transcription and translation signals. The protein was expressed in frame with 6 His residues, which enable a one-step purification of the target protein (on a Nickel-NTA column). The His tag was expressed at the C-terminal of the gene.

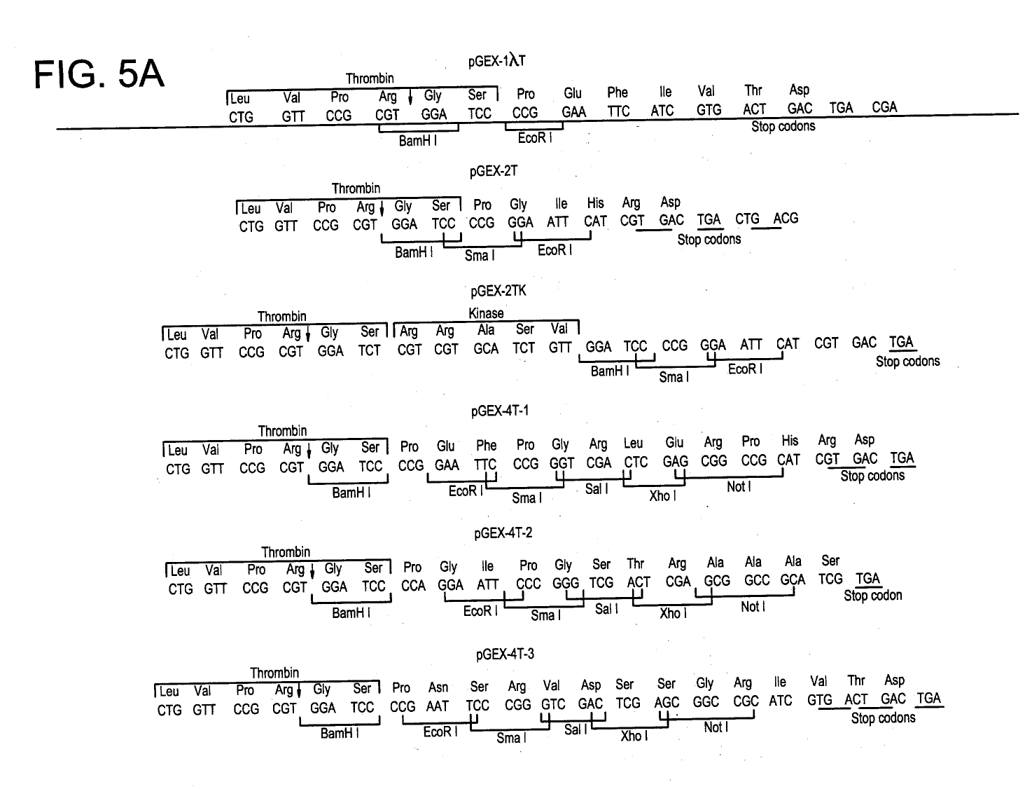

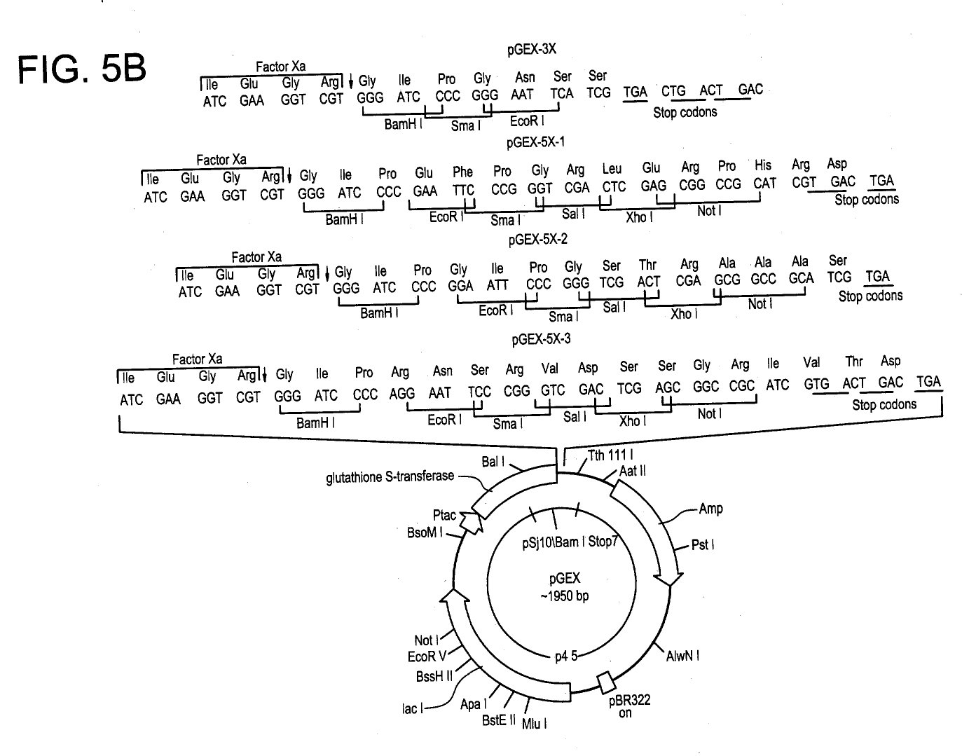

pGEX 5×-2 is a 4.9 kb vector (FIG. 5). The expression of the cloned gene is under the control of tac promoter, which is IPTG inducible and allows high level expression of the cloned genes. The protein was expressed as C-terminal fusion with GST moiety, which enables the purification of the protein on a glutathione-sepharose column.

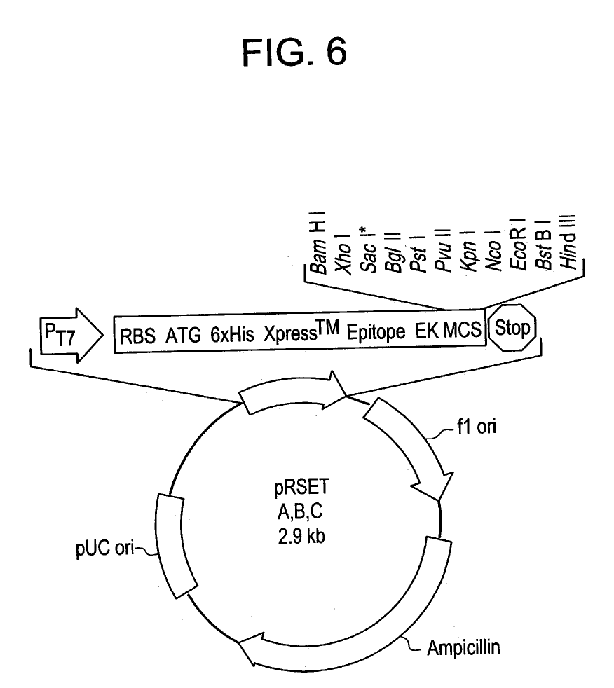

pRSET A is a 2.9 kb vector (FIG. 6). The target genes are cloned under control of strong bacteriophage T7 transcription and translation signals. The protein was expressed in frame with 6 His residues, which enable a one-step purification of the target protein (On a Nickel-NTA column). The His tag is expressed at the N-terminal of the gene.

### EXAMPLE 3

### Cloning of the Multivalent Synthetic Gene

### EXAMPLE 3a

### Cloning of the Multivalent Synthetic Gene in Pet-22b(+) Vector

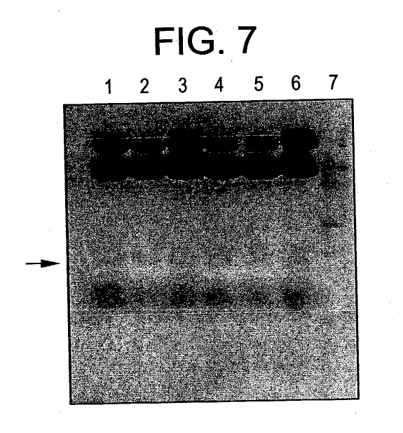

The plasmid pET 22b (+) was digested with the restriction enzymes EcoR1 and Not1, followed by dephosphorylation using Calf-alkaline phosphatase and gel purified to be used as the vector. In a similar manner, the 336 bp synthetic gene product (with the EcoR1/Not1) sites was restricted digested, and gel purified. The vector (pET-22b (+) and the insert (multivalent gene) were ligated in different ratios and transformed in DH5a cells. Plasmid isolation of the transformed colonies was carried out (by alkaline lysis method). The positive clones were identified by Restriction enzyme digestion (using EcoR1 and Not1). As seen in FIG. 7, a large number of plasmid clones showed a fall-out of 336 bp after the restriction enzyme digestion. Nucleotide sequencing was done for these clones using standard sequencing protocols.

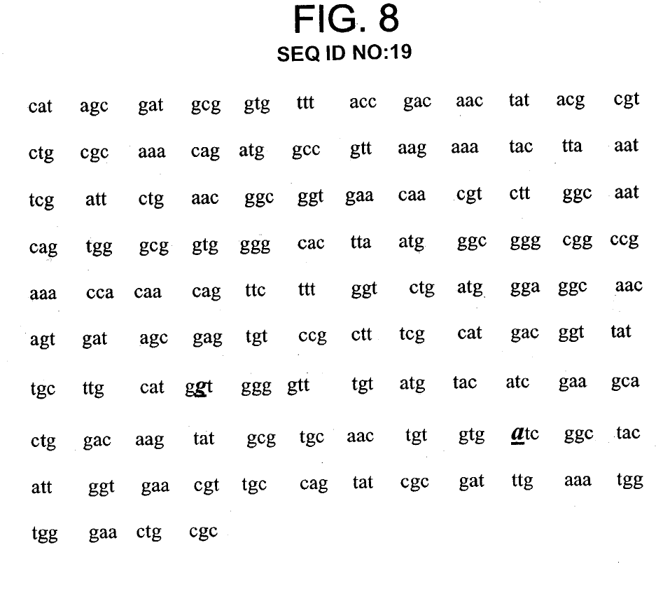

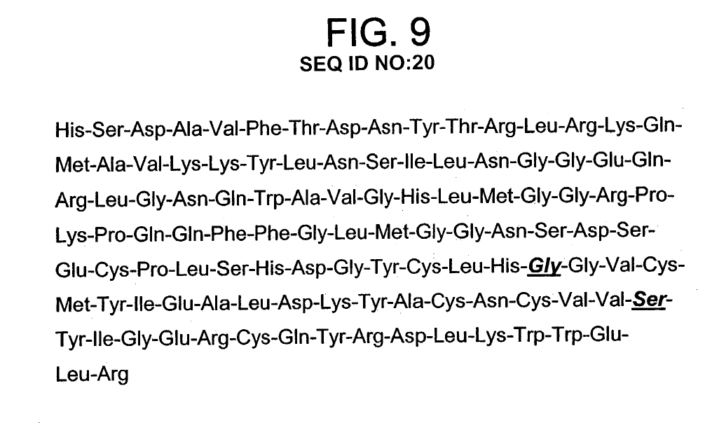

Amongst the clones sequenced, clone #2 (FIG. 8) had 99% identity with the original multivalent sequence. Clone #2 has 2 substitutions towards the C-terminal of the fourth peptide viz. the EGF peptide. (FIG. 9) These substitutions do not effect the immunogenicity or the biological activity of the multivalent peptide as the overall reading frame and the epitopes of the four peptides along with their Gly-Gly linkers have been maintained.

The gene was further expressed and purified on a Nickel-NTA column.

### EXAMPLE 3b

### Cloning of the Multivalent Synthetic Gene in pGEX-5×2 Vector

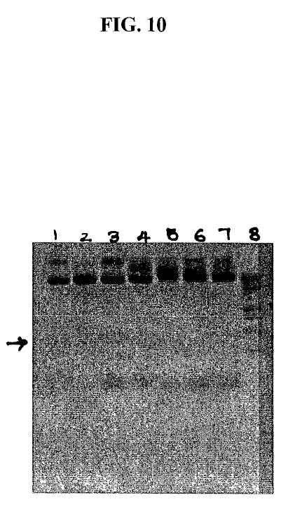

Following a similar strategy, the multivalent gene was cloned at the EcoR1/Not1 site of the pGEX-5×2 vector. The clones were screened by Restriction enzyme digestion. A large number of positive clones were obtained (FIG. 10)

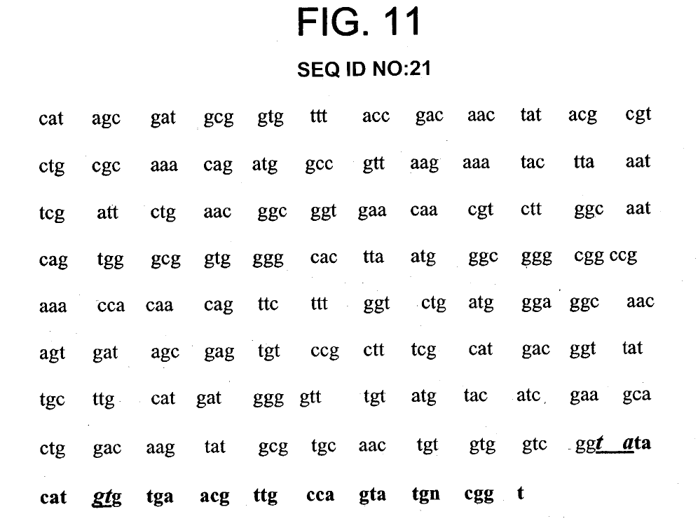

Amongst the clones sequenced, clone #16 (FIG. 11) had 98% identity with the original multivalent sequence. The data shows an addition of a nucleotide towards the 3′ end of the gene. Consequently, there has been a shift in the reading frame, leading to the inclusion of a stop codon at the 99^{th} codon position (FIG. 12). As a result the last 14 aa are not expressed. Also, due to nucleotide substitutions, 3 amino acids prior to the stop codon have been changed. These substitutions do not effect the immunogenicity or the biological activity of the multivalent peptide as the overall reading frame and the epitopes of the four peptides along with their Gly-Gly linkers have been maintained.

The protein was further expressed and purified on a glutathione-sepharose column.

### EXAMPLE 3c

### Cloning of the Multivalent Synthetic Gene

### (With a Stop Codon) in Pet-22b(+) Vector

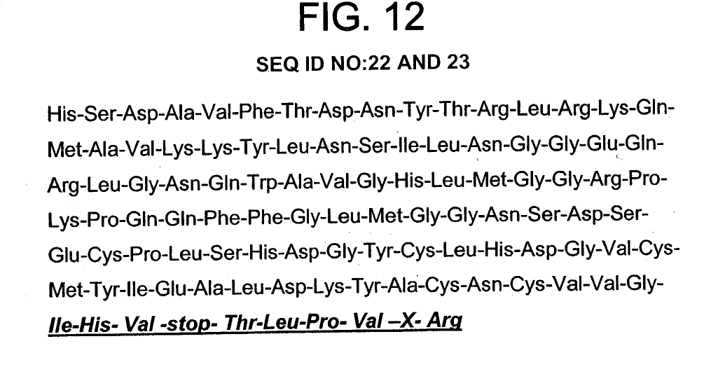

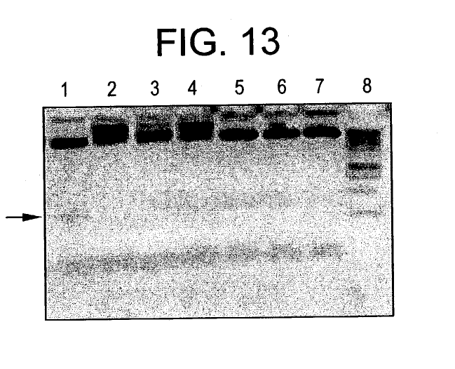

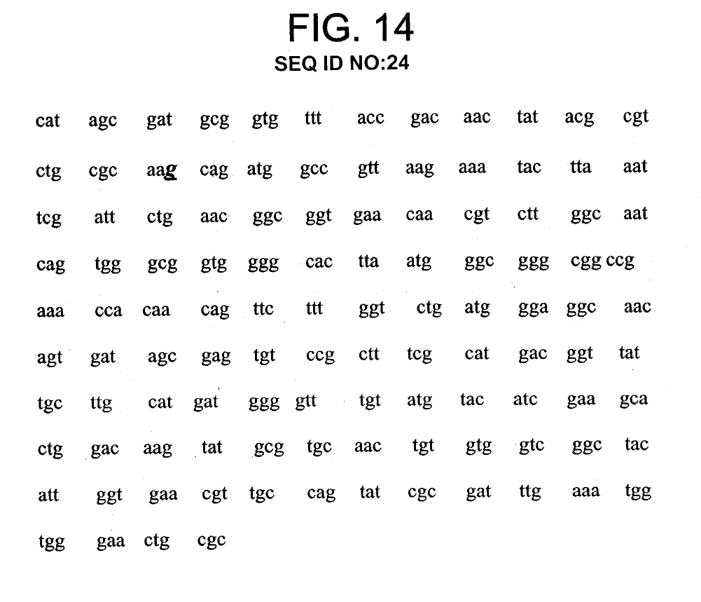

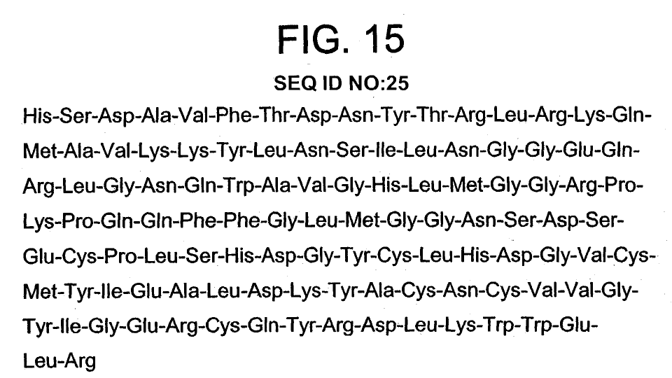

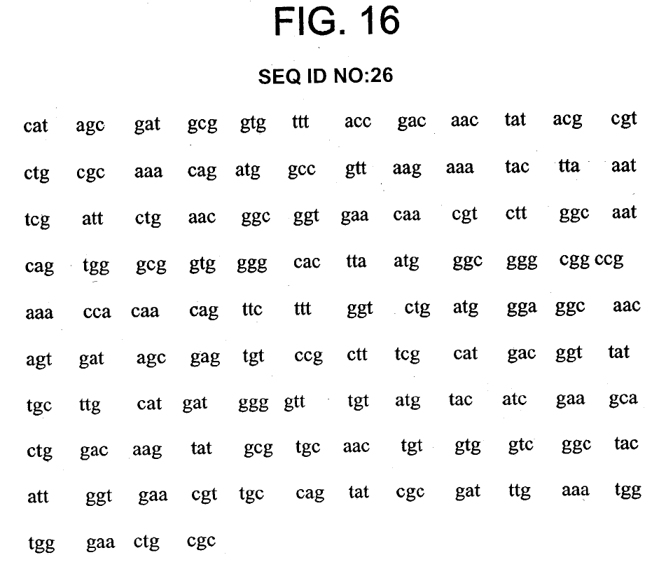

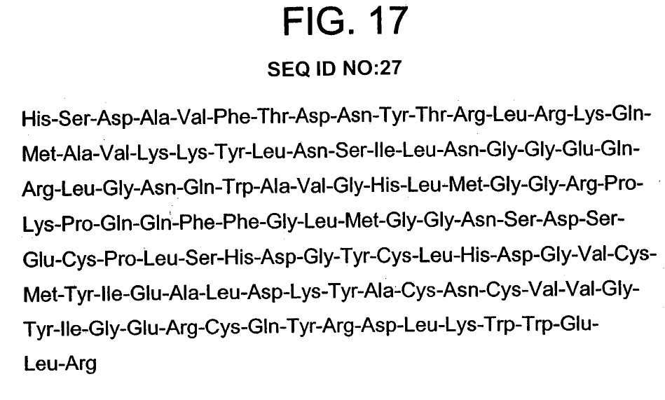

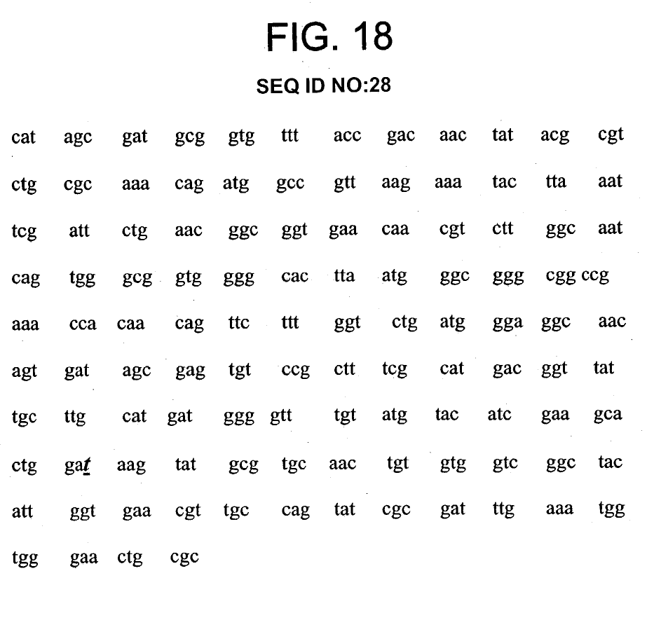

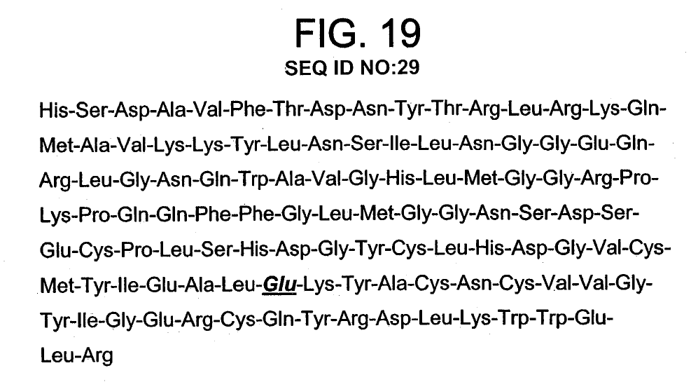

As discussed above, the multivalent gene was successfully cloned in pET-22b (+). However, the vector does not have a protease cleavage site by which the tag can be cleaved off from the full-length mature protein. To overcome this, an alternative strategy was followed. Fresh primers were designed with additional stop codon site in the reverse primer. This enables us to amplify our final multivalent synthetic gene of 336 bp with the stop codon incorporated in it. Using this product a similar strategy of cloning in pET-22b (+) was followed. This would enable us to purify the expressed multivalent protein using standard chromatography procedures. Following cloning and screening, a large number of transformed clones were positive (FIG. 13). Clone #1 (FIG. 14, FIG. 15), 9 (FIG. 16, FIG. 17), and 14 (FIG. 18, FIG. 19) were selected after standard DNA sequencing.

### EXAMPLE 4

### Expression and Purification of the Multivalent Protein

Based on the sequencing data, the following clones were selected for further studies—clone #2 (pET-mv2, with His tag); clone #16 (pGEX-mv16, with GST tag); clone #1, 9 and 14 (pET-SC 1, 9 and 4, respectively with a stop codon immediately after the gene sequence).

### EXAMPLE 4a

### Expression of Clone #2 (Pet-Mv2)

### Induction and Expression of the Protein

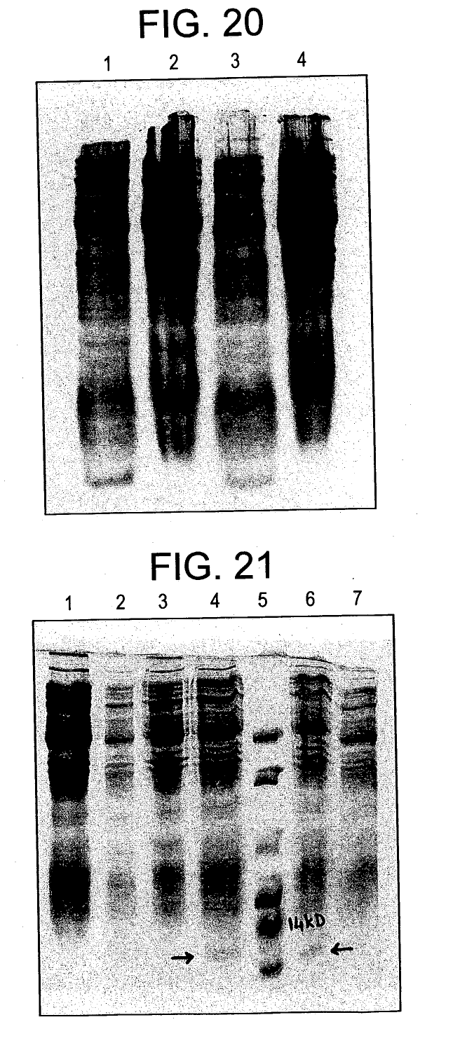

pET-mv2 was transformed in the expression system E. coli BL21DE3λ. Standard procedure of the induction of target protein expression was followed. Induction of protein expression was standardized for two different temperatures, viz. 30° C. and 37° C. For optimizing the temperature and time period of induction, mini-scale protein expression was studied. A single colony was inoculated in 5 ml LB broth (containing 100 μg/ml Ampicillin) and incubated overnight at 37° C./in an incubator shaker with aeration at 220 rpm. Inoculated 5 ml of LB medium containing 100 μg/ml Ampicillin with 100 μl of the overnight culture. Following inoculation, cultures were incubated at 37° C./220 rpm until the optical density of the culture at 600 nm was 0.6. Transferred 1 ml of the uninduced culture (zero-time aliquot) to microfuge tubes. Induced the remainder of the culture by adding IPTG to a final concentration of 1 mM and continued incubation at 30° C. for 3 hours. At various time points during the induction period (eg. ½, 1, 1½, 2, 2½ and 3 hour), transferred aliquot of the cultures (both the induced and the uninduced cultures). After each time point, cells of each culture was pelleted down. Each pellet was resuspended in 100 μl of 1×SDS gel-loading buffer, boiled the samples for 5 min and loaded on a 15% analytical SDS-PAGE gel (FIG. 20). Following a similar methodology, the time-kinetics of induction was carried out at 37° C. for a period of 3 hours and subsequently analyzed on a 15% analytical SDS-PAGE gel (FIG. 21). Comparing FIGS. 27 and 28, it was concluded that the induction of the multivalent protein was optimal at 30° C. with an induction period of 2½ hours.

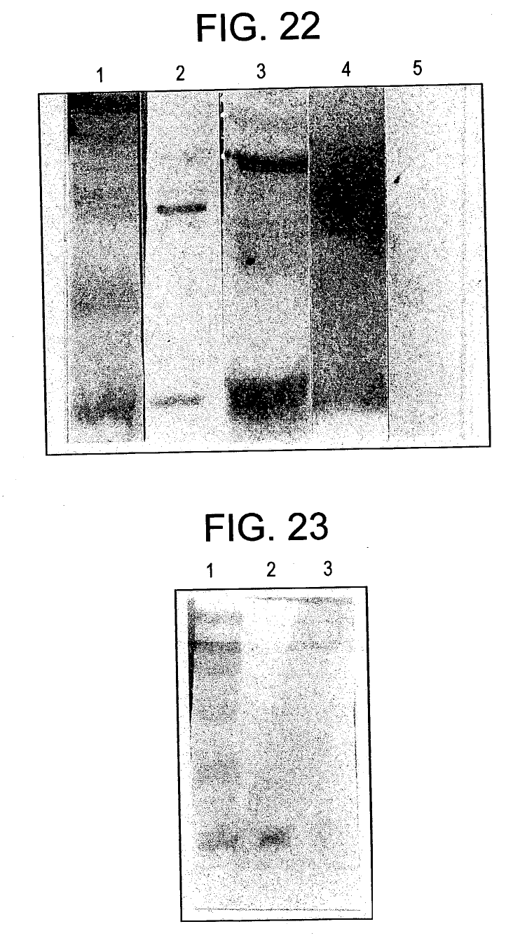

Large-scale induction and expression of the pET-MV2 clone was done. Immunoblot of the crude extract with antibodies against the peptides comprising the multivalent protein viz., anti-VIP, anti-EGF, and anti-Substance-P further confirmed the expression of the multivalent protein. Following SDS-PAGE, the proteins were electrophoretically transferred on the nitrocellulose membrane and non-specific sites blocked with 3% BSA. The membrane was then probed with each of the anti-peptide antibodies, followed by peroxidase conjugated secondary antibodies. Protein band of the expected size was seen in the induced crude extract samples (FIG. 22). As control, uninduced, sample was probed with anti-SP antibody (FIG. 22, lane 5). Immunoblot of the crude extract with anti-His antibodies (FIG. 22, lane 2) ensured the expression of the full-length protein.

### Purification of the Multivalent Protein (with His-Tag)

The expression of the multivalent protein in fusion with a C-terminal poly-histidine tag enables one step purification of the protein on a Ni³⁺-NTA column. The poly-histidine tag is poorly immunogenic and at a pH of 8.0, the tag is small, uncharged, and therefore generally does not affect the secretion, compartmentalization, or folding of the fusion protein within the cell. The protein purification on the Ni²⁺-NTA column (Qiagen) was done under denaturating as well as native conditions.

### Purification Under Native Conditions

The cell pellet was resuspended (from 100 ml culture) in 4 ml of binding buffer [50 mM NaH₂PO₄; 300 m NaCl; 10 mM imidazole; pH 8.0], containing 1×protease inhibitor cocktail and 1% Triton-X-100. The cell solution was sonicated (in ice) for 3 min, followed by centrifugation at 13,000 rpm for 15 min. at 4° C. The Ni-NTA resin was equilibrated with the lysis buffer and this Ni²⁺-NTA slurry was applied to the cell lysate, mixed gently by shaking at 4° C. for 30 min, and then loaded the lysate-Ni-NTA mixture on a column and the flow-through collected. The column was washed thoroughly with the wash buffer [50 mM NaH₂PO₄, 300 m NaCl; 10 mM imidazole; pH 8.0], and the protein eluted with the elution buffer [50 mM NaH₂PO₄, 300 m NaCl; 300 mM imidazole; pH 8.0]. The histidine residues in the 6×H is tag has imidazole ring in the side chain that interacts with the Ni ions on the column. If the imidazole concentration in the elution buffer is increased to 100-300 mM, the 6×His-tagged protein would diassociate because they can no longer compete for binding sites on the Ni-NTA resin. Fractions of 500 μl were collected, and loaded on 15% analytical SDS-PAGE. Following SDS-PAGE, the proteins were electrophoretically transferred on the nitrocellulose membrane whose non-specific sites were blocked with 3% BSA. The membrane was then probed with anti-SP antibody. (FIG. 23).

### Purification Using Standard Chromatography

The cell pellet was resuspended (from 100 ml culture) in 50 mM Tris buffer, pH 8.5. The cell suspension was sonicated (in ice) for 8 cycles 1 minutes/cycle, followed by centrifugation at 12,000 rpm for 10 min. at 4° C. The pellet was resuspended in 50 mM Tris buffer, pH 8.5 containing 2% Deoxycholate. The cell suspension was sonicated (in ice) for 8 cycles, 1 minute/cycle, followed by centrifugation at 12,000 rpm for 10 min. at 4° C. The pellet was resuspended in MilliQ water, and further centrifuged at 12,000 rpm for 10 min. at 4° C. The pellet was solubilized in 100 mM Tris buffer, pH 8.5 containing 2M Urea. The solution was made alkaline by increasing the pH to above 10, centrifuged at 12,000 rpm/10 min/4° C. The supernatant was diluted to 5 times the original volume and pH decreased to 8.5. The sample was loaded on a Q sepharose column (pre-equilibrated with 20 mM Tris, pH 8.5, 2M Urea). The column was washed with 3 volumes of equilibration buffer and eluted with increasing concentration of Nacl (in equilibration buffer-upto 1 M NaCl). The eluted samples were checked on a 15% SDS-PAGE gel.

### Electroelution of the Multivalent Protein

Expression of the pET-MV2 clone results in a full-length multivalent protein with a 6-His tag. The 6 His tag being very small in size does not interfere with the structure and function of the protein and can be used for downstream applications. In a parallel experiment, electroelution of the protein from SDS-PAGE was done. The crude extract of the induced pET-MV2 sample was loaded on a 15% preparative gel. Electrophoresis was done at constant voltage of 100V for about 3 hrs. Two longitudinal strips were cut from the right and left side of the gel, stained with Coomassie blue. After detsaining, the stained side strips were aligned with the rest of the gel and used as guides to cut out bands from the unstained gel.

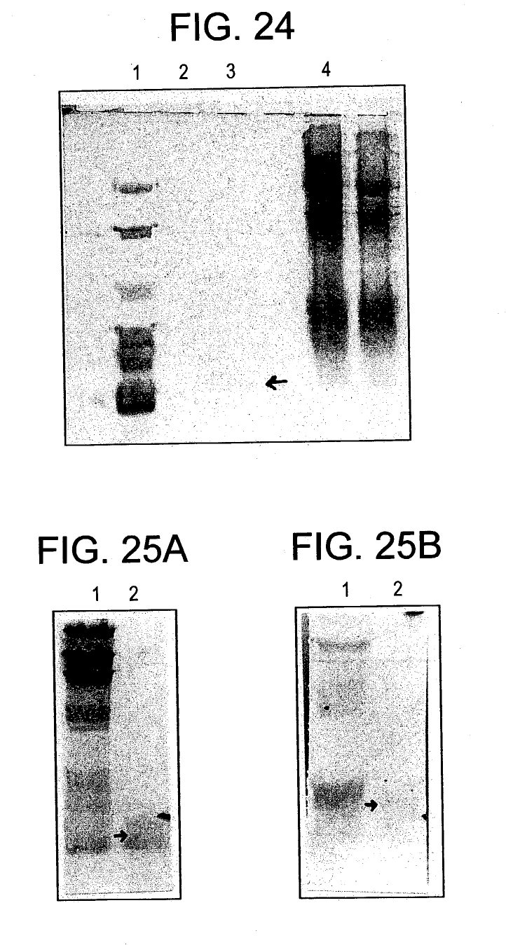

The gel strip was equilibrated in three changes of 2 mM-phosphate buffer, pH 6.8. This removes the toxic components—tris, glycine, SDS, etc. The gel strip was cut into small pieces, sealed in dialysis tubing (M.w. cut off-3,500) with 2 mM phosphate buffer, pH 6.8 to just cover the gel slices. Dialysis tubing was transferred to the platform of horizontal gel electrophoresis apparatus filled with 2 mM-phosphate buffer. Electroelution was carried out at 40 V for 40 min at 4° C. Significant amount of protein sticking to dialysis tubing was desorbed by reversal of current for 1 min. towards the end of main elution period. The protein sample was taken out from the dialysis bag and was concentrated in a lyophilizer, under cold conditions. It was reconstituted in minimal volume of 2 mM PBS and analyzed on a 15% SDS-PAGE gel. The protein moved as a single band of approximately 11 kDa on SD-PAGE (FIG. 24). Immunoblot of the protein with the individual anti-peptide antibodies showed sharp band, a representative with the anti-Substance P antibodies as seen in FIG. 25, Lane 1 and 2.

Here, the multivalent protein was successfully expressed as His tag fusion protein, and was purified by affinity column chromatography as well as directly electroeluted from the gel. This protein is being used for further characterisation studies.

### EXAMPLE 4b

### Expression of Clone #16 (PGEX-Mv 16)

### Induction and Expression of the Protein

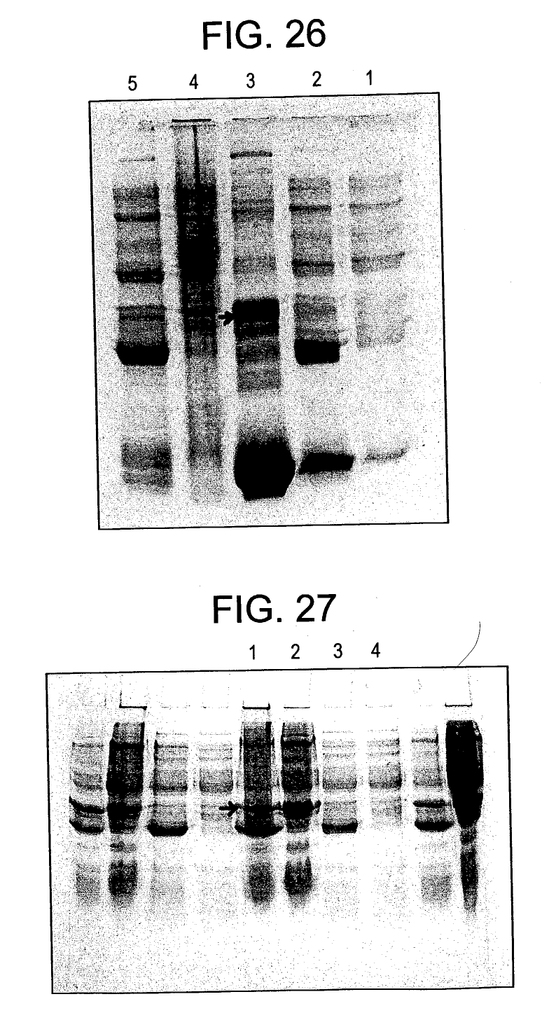

Following a methodology similar to that for pET-mv2, the induction of protein expression was standardized for two different temperatures, viz. 30° C. and 37° C. FIG. 26 and FIG. 27 shows the SDS-PAGE profile of the time-kinetics study. From the profile it was concluded that the induction of the multivalent protein was optimal at 30° C. with an induction period of 2 hours.

Large-scale induction and expression of the pGEX-mv16 clone was done. Immunoblot of the crude extract with anti-peptide antibodies confirmed the expression of the multivalent protein (FIG. 28). As controls, uninduced sample and induced sample of the vector alone was taken.

### Purification of the Multivalent Protein (with GST-Tag)

The expression of the multivalent protein in fusion with a N-terminal GST tag enables one step purification of the protein on a glutathione-sepharose column. Bound GST fusion proteins are readily displaced from the column by elution with buffers containing free glutathione.

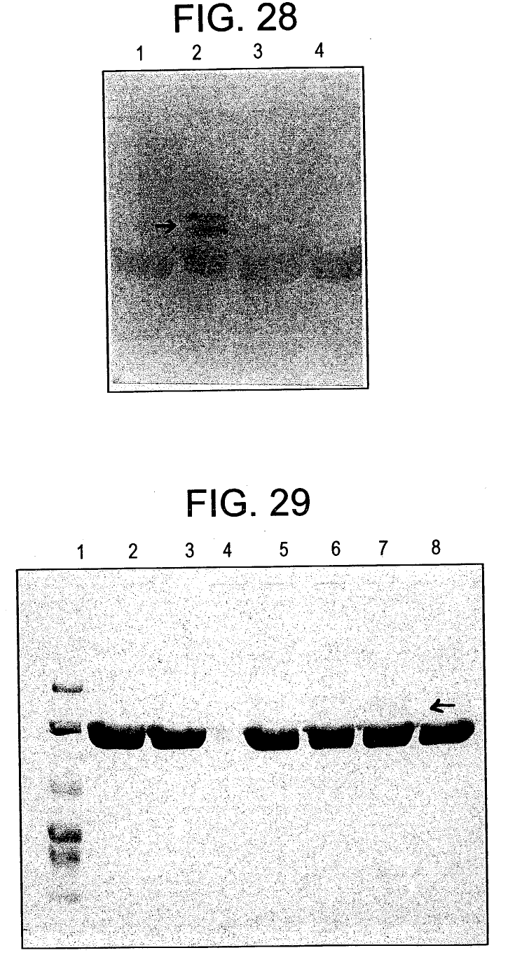

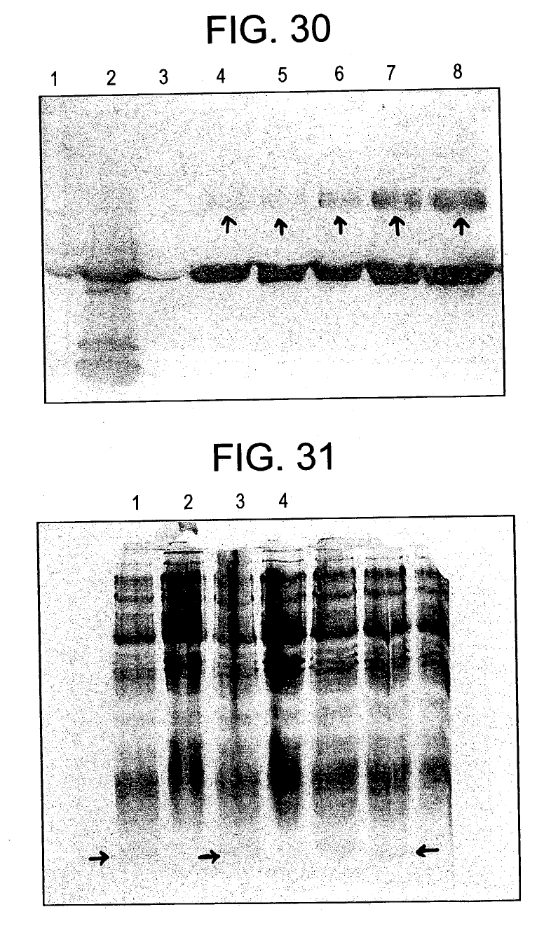

Resuspended the cell pellet (from 100 ml culture) in 4 ml of phosphate-buffered saline, containing 1X protease inhibitor cocktail and 1% Triton-X-100. To the cell suspension lysozyme was added (final concentration of 1 mg/ml) and incubated for 30 min, followed by addition of 10 ml of 0.2% Triton-X-100, with thorough mixing. Next, Dnase and RNase were added to a final concentration of 5 μg/ml, and the incubation was continued for 10 min at 4° C. To the supernatant added DTT to a final concentration of 1 mM. As an alternative to lysozyme treatment, the cell lysis was done by sonicating the cells (in ice) for 3 min. The cell lysate (prepared in either way) was mixed with PBS-equilibrated glutathione-sepharose, and the mixture incubated for 30 min. at room temperature. The lysate-glutathione-resin mixture was then loaded on a column, and the flow-through collected, and column washed thoroughly with PBS. The protein was eluted with the glutathione-elution buffer [20 mM reduced glutathione in 50 mM Tris-HCl (pH 8.0)]. SDS-PAGE analysis of the GST column purified fractions shows faint bands corresponding to the fusion protein (FIG. 29), [cell lysate prepared by sonication of cells] was almost similar. The purified fractions were further characterized by immunoblot using the individual anti-peptide antibodies (FIG. 30).

### EXAMPLE 4c

### Expression of Clones #1, 9 and 14 (Pet-22b Mv16)

### Induction and Expression of the Protein

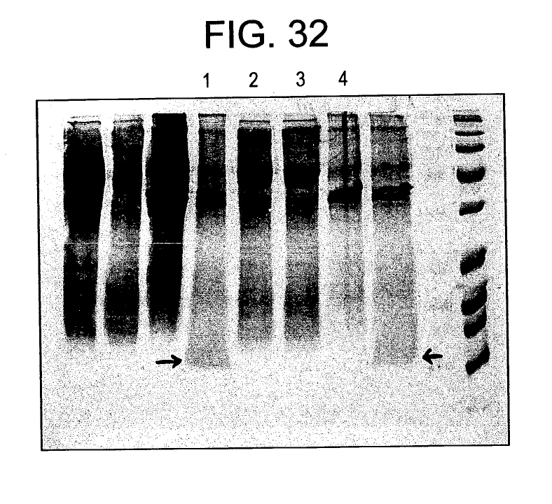

All the three clones, viz. pET-mvsc1, pET-mvsc 9 and pET-mvsc14 were transformed in the expression system E. coli BL21DE3λ. Standard procedure of the induction of target protein expression was followed. Following a methodology similar to that for pET-mv2, the induction of protein expression was standardized for two different temperatures, viz. 30° C. and 37° C. FIG. 31 and FIG. 32 shows the SDS-PAGE profile of the time-kinetics study for the clones pET-mvsc1, and pET-mvsc14 respectively, no protein expression was not seen in clone #9. From the profile it was concluded that the induction of the multivalent protein was optimal at 30° C. with an induction period of 2½ hours for both pET-mvsc1 and pET-mvsc14.

Large-scale induction and expression of the pET-mvsc 1 and 14 was done. Standard conventional purification techniques would be followed for the purification of the protein.

### EXAMPLE 5

### Production of Polyclonal Antibodies in Murine Model

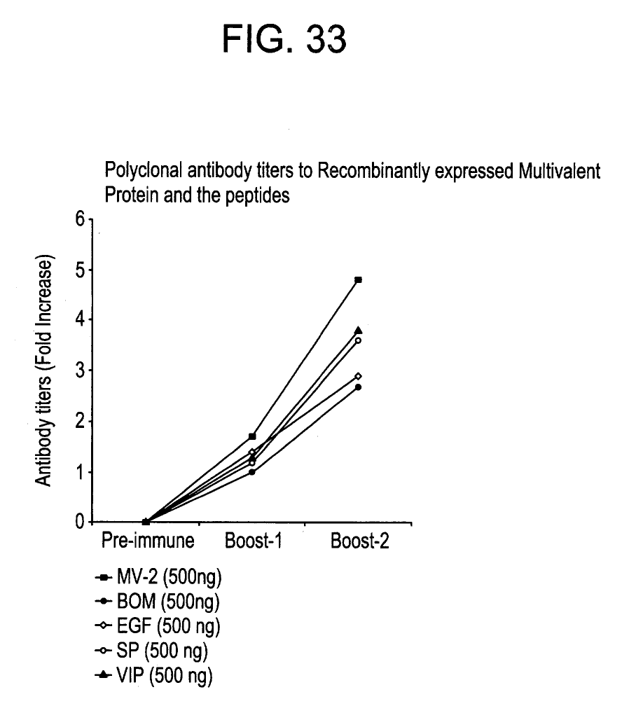

The recombinantly expressed multivalent protein was used to raise polyclonal antibodies in rats. 4-6 weeks old male Wistar rats, obtained from National Institute of Nutrition, Hyderabad were used. On arrival the rats were kept in quarantine for a week before use. After a week, pre-immune blood was collected and rats were immunized by s.c. route at a dose of 15-20 ug/rat with the multivalent protein. Three weeks post immunization a booster dose of the multivalent protein was administered in a similar fashion. Rats were immunized with 15 ug of multivalent protein s.c on the left flank. Two weeks post-boost, the rats were bled from the retro-orbital vein and serum separated. The animals were given a periodic boost of the multivalent protein and bleeds collected. Production of specific immune response to the multivalent protein was observed using standard ELISA technique. The polyclonal antibodies generated in the rats were titrated using ELISA. 96-well ELISA plates were coated with either multivalent protein or each of the individual peptides. (FIG. 33).

### EXAMPLE 6

### Elucidation of Immune Response

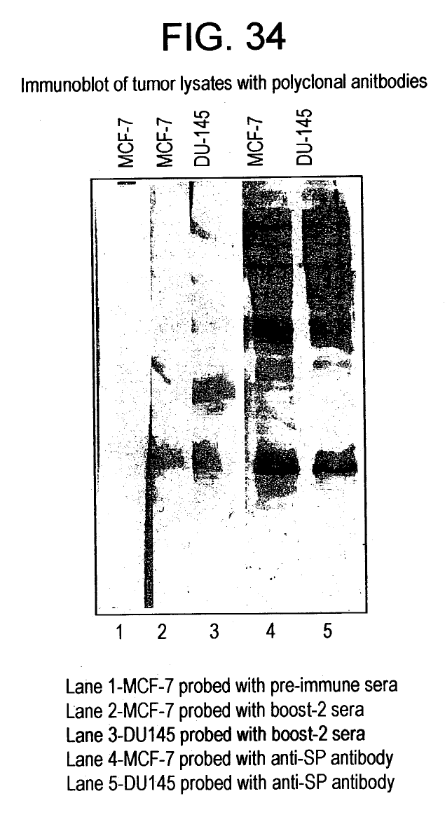

Established cancer cell lines such as DU145 (Prostate cancer) and MCF-7 (Breast cancer) were used to study the immune response to the multivalent polyclonal antibodies, raised in rats. Cells were cultured in vitro using appropriate culture medium and standard growing conditions, to confluency. Cells were trypsinised and 2−3×10⁶ cells were taken for the study. Cells were suspended in 0.1 M PBS and sonicated for 5 minutes. Following sonication, it was centrifuged at 13,000 rpm for 20 minutes at 4° C. The supernatant was collected and used for all further studies. Protein estimation (Bradford reagent) of the culture supernatant was carried out and appropriate amount of the supernatant was used to load on SDS-PAGE gel, followed by Western Blot. The polyclonal antibodies to the recombinantly expressed multivalent protein recognized tumor cell antigens of DU145 and MCF-7 cancer cells, as visualized by immunoblot studies. Further, the tumor cell lyasates were also probed with the commercially available anti-peptide antibodies (FIG. 34).

| | |
|---|---|
| VASOACTIVE INTESTINAL PEPTIDE (VIP) | |
| His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn-NH₂ | SEQ ID NO:1 |
| BOMBESIN (BOM) | |
| Glu-Gln-Arg-Leu-Gly-Asn-Gln-Trp-Ala-Val-Gly-His-Leu-Met | SEQ ID NO:2 |
| SUBSTANCE P (SP) | |
| Arg-Pro-Lys-Pro-Gln-Gln-Phe-Phe-Gly-Leu-Met | SEQ ID NO:3 |
| EPIDERMAL GROWTH FACTOR (EGF) | |
| Asn-Ser-Asp-Ser-Glu-Cys-Pro-Leu-Ser-His-Asp-Gly-Tyr-Cys-Leu-His- | SEQ ID NO:4 |
| Asp-Gly-Val-Cys-Met-Tyr-Ile-Glu-Ala-Leu-Asp-Lys-Tyr-Ala-Cys-Asn- | |
| Cys-Val-Val-Gly-Tyr-Ile-Gly-Glu-Arg-Cys-Gln-Tyr-Arg-Asp-Leu-Lys- | |
| Trp-Trp-Glu-Leu-Arg | |
| Designed Sequence for Synthetic Multivalent Cancer Vaccine | |
| Total number of amino acids=112 | |
| His-Ser-Asp-Ala-Val-Phe-Thr-Asp-Asn-Tyr-Thr-Arg-Leu-Arg-Lys-Gln- | SEQ ID NO:5 |
| Met-Ala-Val-Lys-Lys-Tyr-Leu-Asn-Ser-Ile-Leu-Asn-Gly-Gly-Glu-Gln- | |
| Arg-Leu-Gly-Asn-Gln-Trp-Ala-Val-Gly-His-Leu-Met-Gly-Gly-Arg-Pro- | |
| Lys-Pro-Gln-Gln-Phe-Phe-Gly-Leu-Met-Gly-Gly-Asn-Ser-Asp-Ser-Glu- | |
| Cys-Pro-Leu-Ser-His-Asp-Gly-Tyr-Cys-Leu-His-Asp-Gly-Val-Cys-Met- | |
| Tyr-Ile-Glu-Ala-Leu-Asp-Lys-Tyr-Ala-Cys-Asn-Cys-Val-Val-Gly-Tyr- | |
| Ile-Gly-Glu-Arg-Cys-Gln-Tyr-Arg-Asp-Leu-Lys-Trp-Trp-Glu-Leu-Arg | |
| Nucleotide Sequence of Multi-Valent Vaccine | |
| His Ser Asp Ala Val Phe Thr Asp Asn Tyr | SEQ ID NO:6 |
| CAT AGC GAT GCG GTG TTT ACC GAC AAC TAT | |
| Thr Arg Leu Arg Lys Gln Met Ala Val Lys | |
| ACG CGT CTG CGC AAA CAG ATG GCC GTT AAG | |
| Lys Tyr Leu Asn Ser Ile Leu Asn Gly Gly | |
| AAA TAC TTA AAT TCG ATT CTG AAC GGC GGT | |
| Glu Gln Arg Leu Gly Asn Gln Trp Ala Val | |
| GAA CAA CGT CTT GGC AAT CAG TGG GCG GTG | |
| Gly His Leu Met Gly Gly Arg Pro Lys Pro | |
| GGG CAC TTA ATG GGC GGG CGG CCG AAA CCA | |
| Gln Gln Phe Phe Gly Leu Met Gly Gly Asn | |
| CAA CAG TTC TTT GGT CTG ATG GGA GGC AAC | |
| Ser Asp Ser Glu Cys Pro Leu Ser His Asp | |
| AGT GAT AGC GAG TGT CCG CTT TCG CAT GAC | |
| Gly Tyr Cys Leu His Asp Gly Val Cys Met | |
| GGT TAT TGC TTG CAT GAT GGG GTT TGT ATG | |
| Tyr Ile Glu Ala Leu Asp Lys Tyr Ala Cys | |
| TAC ATC GAA GCA CTG GAC AAG TAT GCG TGC | |
| Asn Cys Val Val Gly Tyr Ile Gly Glu Arg | |
| AAC TGT GTG GTC GGC TAC ATT GGT GAA CGT | |
| Cys Gln Tyr Arg Asp Leu Lys Trp Trp Glu Leu Arg | |
| TGC CAG TAT CGC GAT TTG AAA TGG TGG GAA CTG CGC | |
| Sequences of Designed Oligo′s: | |
| MVSV 1: (1-73) CDS | |
| 5′ CAT AGC GAT GCG GTG TTT ACC GAC AAC TAT ACG CGT | (SEQ ID NO:7) |
| CTG CGC AAA CAG ATG GCC GTT AAG AAA TAC TTA AAT T3′ | |
| MVSV 2: (53-126) NCDS | |
| 5′ CCC CCA CCG CCC ACT GAT TGC CAA GAC GTT GTT | (SEQ ID NO:8) |
| CACCGC CGT TCA GAA TCG AAT TTA AGT ATT TCT TAA | |
| CGG3′ | |
| MVSV 3: (106-179)NCDS | |
| 5′ TTG CCT CCC A TC AGA CCA AAG AAC TGT TGT GGT TTC | (SEQ ID NO:9) |
| GGC CGC CCG CCC A TT AAG TGC CCC ACC GCC CAC TGA T | |
| T3′ | |
| MVSV 4: (159-232) NCDS | |
| 5′ ACC CCA TCA TGC AAG CAA TAA CCG TCA TGC GAA AGC | (SEQ ID NO:10) |
| GGA CAC TCG CTA TCA CTG TTG CCT CCC ATC AGA CCA | |
| AAG3′ | |
| MVSV 5: (212-285) NCDS | |
| 5′ GCC GAC CAC ACA GTT GCA CGC ATA CTT GTC CAG TGC | (SEQ ID NO:11) |
| TTC GAT GTA CAT ACA AAC CCC ATC ATG CAA GCA ATA | |
| AC3′ | |
| MVSV 6: (265-336) NCDS | |
| 5′ GCG CAG TTC CCA CCA TTT CAA ATC GCG ATA CTG GCA | (SEQ ID NO:12) |
| ACG TTC ACC AAT GTA CCC GAC CAC ACA GTT GCA CGC 3′ | |
| MVSV 7: (1-20) CDS | |
| 5′ CAT AGC CAT GCG GTG TTT AC 3′ | (SEQ ID NO:13) |
| MVSV 8: (317-336) NCDS | |
| 5′ CC GCA GTT CCC ACC ATT TCA 3′ | (SEQ ID NO:14) |
| MVSV 9: CDS | |
| 5′ GAA TTC GAA TTC ATG CAT AGC CAT 3′ | (SEQ ID NO:15) |
| MVSV 10: NCDS | |
| 5′ AAT GCG GCC GCT AGC GCA GTT 3′ | (SEQ ID NO:16) |
| MVSV 11: NCDS | |
| 5′ AAT GCG CCC GCC TGA GCG CAG 3′ | (SEQ ID NO:17) |

1. A multivalent vaccine comprising four peptides; vasoactive intestinal peptide, whose sequence is His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn -NH₂ SEQ ID NO: 1), the peptide Bombesin whose sequence is Glu-Gln-Arg-Leu-Gly-Asn-Gln-Trp-Ala-Val-Gly-His-Leu-Met (SEQ ID NO: 2), the peptide Substance P whose sequence is Arg-Pro-Lys-Pro-Gln-Gln-Phe-Phe-Gly-Leu-Met (SEQ ID NO: 3), and the peptide Epidermal growth factor whose sequence is Asn Ser Asp Ser Glu Cys Pro Leu Ser His Asp Gly Tyr Cys Leu His Asp Gly Val Cys Met Tyr Ile Glu Ala Leu Asp Lys Tyr Ala Cys Asn Cys Val Val Gly Tyr Ile Gly Glu Arg Cys Gln Tyr Arg Asp Leu Lys Trp Trp Glu Leu Arg (SEQ ID NO: 4).

2. The vaccine according to claim 1, wherein SEQ ID NO: 1, SEQ ID NO: 2, SEQ ID NO: 3 and SEQ ID NO: 4 are linked together.

3. The vaccine according to claim 1, wherein SEQ ID NO: 1, SEQ ID NO: 2, SEQ ID NO: 3 and SEQ ID NO: 4 are linked together through a linker comprising of at least two amino acids.

4. The vaccine according to claim 3, wherein the linker is Gly-Gly.

5. A vaccine comprising the amino acid sequence of the linked polypeptide consisting of VIP, Bombesin, Substance P and EGF whose sequence is SEQ ID NO: 5.

6. A nucleotide sequence that is optimized to the E. coli codons, whose sequence is SEQ ID NO: 6.

7. A method for SOE ing PCR, whose product is 126 base pairs.

8. A method for SOE ing PCR, whose product is 179 base pairs.

9. A method for SOE ing PCR, whose product is 232 base pairs.

10. A method for SOE ing PCR, whose product is 285 base pairs.

11. A method for SOE ing PCR, whose complete gene sequence is made up of 336 base pairs.

12. A method for SOE ing PCR product of 336 base pairs, with restriction sites EcoR1 and Not1.

13. Clone #16 (pGEX-MV) whose sequence comprises the sequence of claims 1, 2, 3 and 4.

14. Clone #2 (pET-MV) whose sequence comprises the sequence of claims 1, 2, 3 and 4.

15. Clone #1, 9 and 14 (pET-MV: SC) whose sequence comprises the sequence of claims 1, 2, 3 and 4.

16. A recombinantly expressed protein of clone #16.

17. A recombinantly expressed protein of clone #2.

18. A recombinantly expressed protein of clone #1, 9 and 14.

19. The protein as in any one of claims 16 to 18, which is immunogenic and induces a polyclonal antibody response in rats.

20. A multivalent protein according to any one of claims 16 to 18, which is able to generate a T-cell response in the murine model.
